(19) United States
(12) Patent Application Publication
(10) Pub. No.: US 2003/0170643 A1
(43) Pub. Date: Sep. 11, 2003

(54) Regulation of apoB treatment and drug screening for cardiovascular and metabolic disorders or syndromes

(76) Inventors: Edward Fisher, Scarsdale, NY (US); Kevin Jon Williams, Wynnewood, PA (US)

Correspondence Address: NIXON PEABODY LLP, ATTENTION: DAVID RESNICK, 101 FEDERAL STREET, BOSTON, MA, 02110, US

(21) Appl. No.: 10/100,823

(22) Filed: Mar. 18, 2002

Related U.S. Application Data
(63) Continuation-in-part of application No. 09/697,827, filed on Oct. 26, 2000, now abandoned.
(60) Provisional application No. 60/161,537, filed on Oct. 26, 1999.
(60) Provisional application No. 60/276,557, filed on Mar. 16, 2001.
(60) Provisional application No. 60/333,053, filed on Nov. 14, 2001.

(51) Int. Cl. C12Q 1/68; A61K 31/704; A61K 31/455; A61K 31/401; A61K 31/366; A61K 31/355; A61K 31/225; A61K 31/19

(52) U.S. Cl. 435/6; 514/39; 514/355; 514/423; 514/460; 514/548; 514/458; 514/453; 514/571

(57) ABSTRACT

The present invention involves a method of exploiting a novel apolipoprotein-B (apoB) degradation pathway to regulate plasma levels of apoB to treat cardiovascular or metabolic disorders or syndromes, a method of exploiting a novel apolipoprotein-B (apoB) degradation pathway to screen for drugs to treat cardiovascular or metabolic disorders or syndromes, and a method of exploiting a novel apolipoprotein-B (apoB) degradation pathway to screen for genes for diagnosing cardiovascular or metabolic disorders or syndromes.

### CONTINUING APPLICATION DATA

This is a Continuation in Part of application Ser. No. 09/697,827, filed on Oct. 26, 2000, which claims priority to a Provisional Application No. 60/161,537, filed on Oct. 26, 1999. This application also claims priority under 35 U.S.C. §119 based upon U.S. Provisional Patent Application No. 60/276,557 filed on Mar. 16, 2001, and U.S. Provisional Patent Application No. 60/333,053 filed on Nov. 14, 2001

### GOVERNMENT RIGHTS IN THE INVENTION

This invention was made with government support under grants DK 50376, HL 58541, HL 22263, HL 38956, HL 58884 awarded by the National Institutes of Health. The government has certain rights in the invention.

### FIELD OF THE INVENTION

The present invention generally relates to the fields of cardiology and internal medicine and to a method of treating cardiovascular or metabolic disorders or syndromes, as well as a method of screening for drugs to treat such disorders or syndromes. More particularly, the present invention relates to a method of exploiting a novel apolipoprotein B (apoB) degradation pathway to regulate plasma levels of apoB to treat cardiovascular or metabolic disorders or syndromes, to a method of exploiting a novel apoB degradation pathway to screen for drugs to treat cardiovascular or metabolic disorders or syndromes, and to a method of exploiting a novel apoB degradation pathway to screen for genes for diagnosing cardiovascular or metabolic disorders or syndromes.

### BACKGROUND OF THE INVENTION

Blood cholesterol is classified according to the density of its associated lipoproteins. The lipoprotein classes include very low density lipoproteins (VLDL), low density lipoproteins (LDL) and high density lipoproteins (HDL). The corresponding cholesterol classes are VLDL-, LDL-, and HDL-cholesterol, respectively.

Apolipoproteins AI (apoAI) and apolipoprotein B (apoB) are proteins that associate specifically with particular blood lipids. ApoAI associates specifically with HDL-cholesterol, the so-called “good” cholesterol. ApoB associates with VLDL-cholesterol and LDL-cholesterol, the so-called “bad” cholesterols.

ApoB is synthesized primarily by hepatic and intestinal cells. ApoB message level and translational rate in hepatic cells are largely constitutive, and so secretory control is achieved primarily through co- and post-translational degradation of the protein (Yao, Z., et al., (1997) J Lipid Res 38(10), 1937-53 and Davis, R. A. (1999) Biochim Biophys Acta 1440(1), 1-31).

Two specific mechanisms for the destruction of newly synthesized apoB in hepatic cells have been characterized (FIG. 17). The first is endoplasmic reticulum(ER)-associated degradation (ERAD). Newly synthesized apoB in the ER is initially complexed with small amounts of lipid that is shuttled to the apoB by the microsomal triglyceride transfer protein (MTP) (Berriot-Varoqueaux, N. et al., (2000) Annu Rev Nutr 20, 663-697). During conditions of severe lipid deprivation or MTP deficiency, this initial lipidation fails, and the apoB becomes ubiquitinylated, which targets it for degradation by proteosomes.

The second mechanism for degradation of newly synthesized apoB is the re-uptake pathway. Re-uptake occurs after fully assembled apoB-containing particles have been exported across the plasma membrane, but before they have diffused away from the vicinity of the cell by traversing the unstirred water layer that is adjacent to the plasma membrane (Williams, K. J., Brocia, R. W., and Fisher, E. A. (1990) J Biol Chem 265, 16741-16744). A substantial percentage of these nascent apoB-containing particles bind cell-surface receptors, such as LDL receptors or specific heparan sulfate proteoglycans (HSPG), that then bring them back into the cell. Delivery to lysosomes and proteolytic degradation follows. The pathway is stimulated by sterol deprivation, which induces LDL receptor expression, or by the presence of molecules that can bridge between apoB-containing particles and cell-surface proteoglycans.

High cholesterol level is often associated with metabolic or cardiovascular disorders, such as, hypercholesterolemia, hyperlipidemia, diabetes, insulin resistance, atherosclerosis, restenosis, stroke, and coronary artery disease, to name a few. Current therapy in lowering cholesterol has focused on apoB metabolism in the plasma, such as the statin drugs, 3-hydroxy-3-methylglutaryl coenzyme A (HMG-CoA) reductase inhibitors. According to survey data from the National Heart, Lung, and Blood Institute, each year, there are about 36 million individuals that require medication to lower their cholesterol level. Among the 36 million, 20% of them do not respond to statins. In addition, statins cause muscle damage in certain patients. Thus, there is a need to develop a new class of cholesterol lowering drugs that target a different pathway than the current available medicines. It is also desirable to provide a combination therapy by using two classes of medicines that target different pathways.

Since apoB plays a role in maintaining VLDL- and LDL-cholesterol in the bloodstream, it is thought that by reducing secretion of apoB into the bloodstream, the amount of bad cholesterol retained in the bloodstream also can be reduced. Attempts have been made to use MTP inhibitors in reducing secretion of apoB. However, MTP inhibitors often cause severe side effects, such as, fatty liver.

The present invention, for the first time, discloses a third degradation pathway for aopB in hepatic cells, in which the degradative process occurs between the other two, that is, after lipidation in the ER, but before export across the plasma membrane. It is named post-ER pre-secretory proteolysis (PERPP). PERPP can be triggered by Ω-3 fatty acids, also known as fish oils. Importantly, PERPP does not act via microsomal lipid transfer protein inhibition, active proteosomes, cell-surface LDL receptors, cell-surface HSPGs, or functioning lysomsomes. The target particle is a large and relatively mature lipoprotein that contains substantial amounts of lipid and other apoproteins, such as apoE and apoC. Phosphoinositide 3-kinase (PI-3 kinase) activation and lipid peroxidation also are involved.

It is, therefore, an objective of the present invention to provide a novel method of treating cardiovascular or metabolic disorders or syndromes using a compound that stimulates PERPP, as well as a method of screening for drugs that stimulate PERPP to treat such disorders or syndromes. It is also an objective of the present invention to provide a method of screening for genes for diagnosing cardiovascular or metabolic disorders or syndromes associated with defects in PERPP. It is further an objective of the present invention to provide a method of treating HIV-infected patients and a method of screening for HIV protease inhibitors.

Abbreviations

“AA” means “arachidonic acid”.

“ANOVA” means “analysis of variance”.

“ApoB” means “apolipoprotein B”.

“BFA” means “Brefeldin A”.

“BHT” means “butylhydroxytoluene”.

“CE” means “cholesteryl ester”.

“DFX” means “desferrioxamine”

“COX” means “cyclooxygenase”.

“DHA” means “docosahexaenoic acid”.

“EPA” means “eicosapentaenoic acid”.

“ER” means “endoplasmic reticulum”.

“ERAD” means “ER-associated degradation”.

“FA” means “fatty acids”.

“FBS” means “fetal bovine serum”.

“FCHL” means “familial combined hyperlipidemia”.

“HepG2” means “human hepatocarcinoma”.

“HDL” means “high density lipoprotein”.

“HMG-CoA” means “3-hydroxy-3-methylglutaryl coenzyme A”.

“LAC” means “lactacystin”

“LDL” means “low density lipoprotein”.

“MA” means “fatty acid myristic”.

“McA” means “McArdle RH7777”.

“MTP” means “microsomal triglyceride transfer protein”.

“MTPI” means “microsomal triglyceride transfer protein inhibitor”.

“OA” means “oleic”.

“PC” means “phosphatidylcholine”.

“PE” means “phosphatidylethanolamine”.

“PERPP” means “post-ER pre-secretory proteolysis”.

“PI-3 kinase” means “Phosphoinositide 3-kinase”

“POPC” means “1-palmitoyl-2-oleoyl phosphatidyl choline”.

“RPMI” means

“SDS-PAGE” means “SDS-polyacrylamide gel electrophoresis”.

“TG” means “triglyceride”.

“Vit E” or “VE” means “vitamin E”.

“VLDL” means “very low density lipoprotein”.

DEFINITIONS

“Derivative” or “analog,” as used herein, is a derivative or modification of the native original molecule. Derivatives and analogs can be prepared using techniques and principles well-known in the art. The various properties of a molecule that can be modified during the course of analog preparation include, but are not limited to, ionic interactions, hydrogen bonding capability, hydrophobicity, stacking interactions, Van der Waals or steric interactions with the host, and reorganization of the molecule into a conformation resembling the bound conformation of the natural ligand.

“Mutation,” as used herein, means any detectable change in genetic material, e.g. DNA. This includes, but is not limited to, nucleotide(s) substitution, deletion, and insertion.

“Mutation affecting at least one gene”, as used herein, means any detectable change in genetic material, e.g. DNA, that results in elevated or reduced expression of the at least one gene, or the activity of the encoded RNA(s) protein(s).

“Patient,” as used herein, can be one of many different species, including but not limited to, mammalian, bovine, ovine, porcine, equine, rodent and human.

“Substantially” “affect”, “impair”, “reverse”, “decrease”, or “increase,” as used herein, means a 2 fold change, i.e., a more than 50% increase or a <50% decrease.

### BRIEF DESCRIPTION OF THE FIGURES

FIG. 1 illustrates that DHA inhibits the secretion of apoB₄₈ on VLDL, while denser apoB₄₈-containing particles are relatively unaffected. Rat primary hepatocytes were incubated at 37° C. for 4 hr with either OA or DHA (0.8 mM, complexed to 0.16 mM BSA) in the presence of [³⁵S]methionine. Conditioned media samples were subjected to density gradient fractionation, and the labeled apoB₄₈ content of each density class was determined by immunoprecipitation followed by SDS-PAGE and then scintillation counting of the excised gel bands -containing apoB₄₈. Results shown are the mean ±SE (n=6).

FIG. 2 illustrates that DHA inhibits the secretion of apoB₁₀₀ on VLDL, while denser apoB₁₀₀-containing particles are relatively unaffected. HepG2 cells were treated and their conditioned media samples analyzed as in FIG. 1, except that the excised gel band contained apoB₁₀₀. Results shown are the mean ±SE (n=6).

FIG. 3 illustrates that Ω-3 fatty acids inhibit the secretion of total labeled apoproteins associated with VLDL. Rat primary hepatocytes were incubated 4 hr with OA, EPA, or DHA (0.8 mM, complexed to 0.16 mM BSA) or with BSA alone (0.16 mM) in the presence of [³⁵S]methionine. Conditioned media samples were subjected to density gradient fractionation and the total labeled apoprotein contents of the VLDL fractions were quantified by TCA precipitation followed by scintillation counting. After normalization to mg of cell protein, the secretion of labeled apoproteins in the presence of the fatty acid/BSA complexes relative to the secretion in the presence of BSA alone was calculated. Results shown are the mean ±SE (n=6).

FIG. 4 illustrates that the effects of EPA or DHA on the secretion of different labeled apoprotein species associated with VLDL. Rat primary hepatocytes were treated as in FIG. 3. Conditioned media samples were subjected to either: Panel A: density gradient fractionation to isolate VLDL, and the labeled apoprotein species were separated by SDS-PAGE (The resulting fluorogram shows the results from duplicate wells. The migration of the apoprotein size standards is indicated on the right); or, Panel B: immunoprecipitation/SDS-PAGE analysis using an anti-rat apoE antiserum. The cell treatments are BSA (lane 1), OA (lane 2), EPA (lane 3), and DHA (lane 4). Based on densitometry, the average recovery of apoE in the Ω-3 lanes is ˜43% of that in the BSA and OA lanes.

FIG. 5 illustrates that Ω-3 lipids are good substrates for MTP-mediated lipid transfer. HepG2 cells were incubated for 14 hr at 37° C. with ¹⁴C-labeled oleate (OA) or ¹⁴C-labeled EPA complexed to BSA (final concentration of either fatty acid was 0.8 mM). Total lipids were extracted from the washed cell monolayers and separated by preparative TLC into triglyceride (TG), cholesteryl ester (CE), phosphatidylcholine (PC), and phosphatidylethanolamine (PE). The isolated lipid classes were then incorporated (to a content of 0.5 mol %) into vesicles that were used as donors in lipid transfer assays with recombinant MTP. Results were normalized to the transfer of [¹⁴C]triolein and are shown as mean values ±SE (n=4).

FIG. 6 illustrates that Ω-3 lipids do not inhibit the transfer of non-Ω-3 lipids by MTP. Artificial donor vesicles were prepared in which PC that contained oleate esterified at the sn-2 position was replaced with an increasing mole fraction of PC that contained DHA or EPA groups. The transfer by purified bovine MTP of either labeled triolein or labeled POPC to acceptor vesicles was measured in a lipid transfer assay. Results shown are the amounts of labeled lipids transferred relative to the transfer observed with donor vesicles containing no Ω-3 acyl chains and are expressed as mean values ±SE (n=4).

FIG. 7 illustrates that inhibition of MTP reduces the secretion of newly synthesized apoB₁₀₀ without affecting apoE. Rat McA hepatoma cells were incubated at 370C for 4 hr with [ ³⁵S]methionine in the absence (Control) or presence (MTPI) of an inhibitor of MTP activity. Equal aliquots of the conditioned media samples from duplicate wells were subjected to separate immunoprecipitations with anti-apoB or anti-apoE antiserum. For each well, the resulting pellets from the two immunoprecipitations were combined and analyzed by SDS-PAGE and fluorography.

FIG. 8 illustrates that EPA stimulates apoB degradation even when proteasomes are inhibited. Panel A: Rat McA hepatoma cells were incubated at 37° C. for 4 hr in [³⁵S]methionine-containing medium supplemented with either BSA (C) or EPA/BSA complexes (EPA) in the absence (EPA) or presence (EPA+LAC) of the proteasome inhibitor, lactacystin. Samples of cell lysates and conditioned media were subjected to immunoprecipitation analysis with anti-apoB antiserum, followed by SDS-PAGE and fluorography. In three separate experiments, the effect of lactacystin on EPA-induced degradation averaged less than 20%. Panel B: Rat McA hepatoma cells were incubated at 37° C. for 4 hr in [³⁵5]methionine-containing medium supplemented with either BSA (Control) or BSA and lactacystin (LAC). After immunoprecipitation/SDS-PAGE analysis of the cell lysate and media samples, the resulting fluorograms were analyzed by phosphorimager and the recovery of total (cell+medium) apoB₁₀₀ was quantified. The results shown (mean total recovery relative to control, ±SE) are based on 8 independent determinations. * indicates P<0.001, LAC vs. control.

FIG. 9 illustrates that DHA inhibits the secretion of newly synthesized VLDL-apoproteins even when re-uptake is blocked. Rat primary hepatocytes were treated as in FIG. 1, except that media in the indicated wells also was supplemented with heparin (10 mg/ml). Total labeled VLDL-apoproteins were determined as in FIG. 3. The results for the fatty acids are expressed relative to the corresponding result for BSA and are shown as mean ±SE (n=6).

FIG. 10 illustrates the recovery of ER and Golgi-associated apoB₁₀₀ from cells treated with BSA, OA or DHA. Rat primary hepatocytes were incubated for 4 hr in DMEM containing [³⁵S]methionine (300 μCi/ml) and one of the following: (a) 0.16 mM BSA; (b) 0.8 mM OA complexed with 0.16 mM BSA; or (c) 0.8 mM DHA complexed with 0.16 mM BSA. After homogenization of the cells, post-nuclear supernatants were prepared and separated on sucrose gradients (Methods). Panel A shows the distribution of the Golgi and ER markers, a-mannosidase II and calnexin, respectively. Panel B shows the recovery of apoB₁₀₀ from the Golgi (fractions #7-11; filled columns) and ER (fractions #12-20; open columns) regions of the gradient.

FIG. 11 illustrates that brefeldin A increases the total recovery of newly synthesized apoB₁₀₀ from DHA-treated cells. Rat hepatoma cells were incubated at 37° C. for 4 hr in [³⁵S]methionine-containing medium supplemented with either DHA complexed to BSA (DHA) or DHA/BSA plus 4 mg/ml BFA (DHA+BFA). The total recovery of labeled apoB₁₀₀ was determined by immunoprecipitation of cell lysates and media samples, followed by SDS-PAGE and phosphorimager analysis. The data were normalized to total labeled protein in cell lysate plus media, determined using TCA precipitation. The results shown are mean ±SE (n=4).

FIG. 12 illustrates that wortmannin increases the recovery of apoB mass secreted from DHA-treated cells. Rat primary hepatocytes were incubated 5 hr in medium containing either BSA alone (Control), DHA/BSA (DHA), or OA/BSA (OA) and in the absence (−) or presence (+) of 1 μM wortmannin. The recovery of total apoB mass in the conditioned medium samples was determined by radioimmunoassay. Results shown are the mean ±SE (n=5). ANOVA (P<0.0001 for equality of means) was followed by the Dunnett q′ statistical test to detect differences compared to the control group (BSA without wortmannin). *indicates P<0.01.

FIG. 13 illustrates that either an iron chelator (DFX) or lipid antioxidants block DHA-stimulated apoB degradation and reactive oxygen species (ROS) production. Rat hepatoma cells were plated in culture wells and pre-incubated in media containing either DHA or DHA with one of the indicated compounds. Radiolabeled methionine/cysteine was added to the culture media for 15 minutes, after which time, the culture media were replaced by fresh media identical in composition except that radiolabeled methionine/cysteine were replaced by unlabeled methionone/cysteine. The recovery of labeled apoB₁₀₀ ([³⁵S]-apoB₁₀₀) from the media and cell lysate was determined at the indicated time points (with time 0 being the point when the fresh media were applied to the cells) by immunoprecipitating apoB species with a rabbit polyclonal antibody to rat apoB, resuspending the immunoprecipitates, combining aliquots from cell lysate and media samples from each well, resolving the protein contents of the combined immunoprecipitates by SDS-polyacrylamide gel electrophoresis (SDS-PAGE), and exposing the resultant gels to X-ray film in order to visualize the labeled apoB bands. In panel A are shown representative signals for radiolabeled apoB₁₀₀ in each of the treatment conditions. Note that by 120 minutes, the recovery of labeled apoB₁₀₀ from DHA-treated cells was significantly increased by co-treatment with any of the indicated compounds, demonstrating decreased degradation of apoB₁₀₀ when the iron chelator (desferrioxamine; DFX) or anti-oxidants (vitamin E, BHT) were used in combination with DHA. For each treatment, there were at least 2 wells/treatment for each time point and the entire experiment repeated. The signal intensities of the apoB₁₀₀ bands were quantified by densitometry and the mean relative recoveries (the % of the 15 minute, or initial, value still remaining at 120 minutes) ±SEM are shown in panel B. Note that the visual impressions observable in panel A are confirmed by the quantitative analysis; i.e., there is a substantial increase in labeled apoB₁₀₀ recovery with any of the co-treatments. In panel C, the ROS content of the cell lysates at the end of the experiment was measured by the standard TBARS assay (Stocks et al., 1974, Clin Sci Mol Med 47,215-222). Note that compared to control (BSA), DHA induced a large increase in TBARS, as expected because of its high content on unsaturated bonds. Also as expected, the iron chelator and antioxidants prevented the DHA-induced increase in ROS and a comparison of the results in panels B and C shows an inverse relationship between apoB₁₀₀ recovery and TBARS.

FIG. 14 illustrates that DHA, but not saturated MA, induces apoB₁₀₀ degradation in rat hepatoma cells. An experiment similar to the one described in FIG. 13 was performed, with the following changes: A) The immunoprecipitates from cell lysate and media samples were not combined, but separately analyzed. B) In addition to the 15 and 120 minute time points, apoB recovery was determined at 30 minutes. C) In addition to DHA, the effects of the fatty acid myristic (MA) were also tested because it has no unsaturated bonds and should not give rise to ROS. (as confirmed by the TBARS assay shown in FIG. 15, bottom). Note the decreased cell lysate and medium recovery of labeled apoB₁₀₀ from DHA-treated cells, but not in the control (BSA) or MA-treated cells, indicating that MA, in contrast to DHA, did not induce apoB₁₀₀ degradation.

FIG. 15 illustrates that AA stimulates apoB₁₀₀ degradation and increases ROS. An experiment similar to the one described in FIG. 13 was performed with the following changes: A) the polyunsaturated fatty acid arachidonic acid (AA) was used to treat cells, in which AA is well known to induce ROS formation; and B) the ROS contents of cell lysates after treatments with BSA, MA, OA (oleic acid, a mono-unsaturated fatty acid has been shown to induce apoB₁₀₀ degradation), AA, and DHA were determined and compared. Note in the upper panel that the combined recovery (from two separate wells/time point) of radiolabeled apoB₁₀₀ decreased from 15 to 120 minutes, indicating significant apoB₁₀₀ degradation had occurred in the presence of AA. As shown in panel B, this induced degradation was associated with an increase in ROS (as measured by the TBARS assay; as also shown the ROS level in AA treated cells was comparable to those in DHA treated cells). Note that the treatments that do not induce apoB₁₀₀ degradation (BSA, MA, OA) were not associated with increased ROS. In addition to being a source of ROS, AA can also be converted by cyclooxygenase (COX) to bioactive prostaglandin compounds. We therefore repeated the experiment with AA but with co-treatment by the COX inhibitor aspirin. AA still induced apoB₁₀₀ degradation, thereby confirming that its effects on apoB₁₀₀ were mediated by the increase in ROS, not by a COX-dependent pathway.

FIG. 16 illustrates that ROS regulates basal apoB degradation in primary hepatocyes. An experiment similar to the one described in FIG. 13 was performed with the following changes: A) rat primary hepatocytes were studied. B) the treatment conditions were control (BSA) with or without co-treatment with DFX or vitamin E. Note that in the control state, there is approximately a 50% recovery of radiolabeled apoB100 that is associated with a detectable level of ROS (TBARS assay, below). When the ROS level was reduced by either co-treatment, approximately 90% of the apoB₁₀₀ was recovered, indicating a dramatic reversal of the basal level of degradation. These results indicate that the basal level of ROS is an important regulator of apoB₁₀₀ production in primary hepatocytes and that the quantitative contribution of the proteosomal-mediated ERAD pathway must be small (˜10%) relative to the PERPP pathway, which is not mediated by proteosomes, but is regulated by ROS.

FIG. 17 illustrates a schematic diagram of two previously known mechanisms for destruction of newly synthesized hepatocyte apoB, plus the newly discovered pathway, as disclosed herein. These methods of degradation are ERAD, PERPP, and re-uptake.

### DETAILED DESCRIPTION OF THE INVENTION

The present invention relates to a newly discovered apolipoprotein degradation pathway, more particularly, post-ER pre-secretory proteolysis (PERPP), methods of identifying compounds that stimulates PERPP, and methods of treating patients in need thereof by administering one or more compounds that stimulate PERPP to reduce plasma concentration of cholesterol, apoB, and/or a component(s) of an atherogenic lipoprotein.

The present invention provides evidence of the important role of the PERPP pathway in regulating the apoB secretion by hepatic cells. As disclosed in the Methods and Results sections, the degradation of apoB regulated by PERPP occurs in a post-ER compartment or compartments, thus the apoB level in ER is not substantially affected by this pathway. It is demonstrated that the process involves at least one of PI-3 kinases and lipid peroxidation, which leads to modifications of apoB and its destruction by a novel proteolytic mechanism. Also disclosed is that under physiologically relevant conditions, ERAD is close to zero, and the basal degradation under these conditions is mediated by PERPP with a small contribution from re-uptake. Furthermore, PERPP may contribute to the pathogenesis of familial combined hyperlipidemia (FCHL), syndrome X, hyperlipidemia, insulin resistance seen after administration of HIV protease inhibitors, and/or other metabolic syndromes.

One embodiment of the present invention provides methods of screening for a pharmaceutical compound that stimulates PERPP, which methods comprise the steps of, a) contacting hepatic cells with the compound; and b) measuring cellular content and/or secretion levels of an apoB, an apoB-containing lipoprotein and/or a component of an atherogenic lipoprotein to determine the efficacy of the compound, more particularly, the efficacy of stimulating PERPP, in combination with at least one second assay. The second assay includes, but is not limited to, 1) determining that a proteosome inhibitor does not substantially impair the efficacy of the compound; 2) determining that heparin does not substantially impair the efficacy of the compound; 3) determining that the efficacy of the compound is substantially reversible by an antioxidant; and 4) determining that the efficacy of the compound is substantially reversible by a PI-3 kinase inhibitor.

More particularly, the assay of determining that a proteosome inhibitor does not substantially impair the efficacy of the compound includes the steps of pre-incubating the hepatic cells in the presence of the proteosome inhibitor for about 0.5-1 hr, continuing incubation of the hepatic cells with the proteosome and the compound, measuring cellular content and/or secretion levels of apoB, an apoB-containing lipoprotein, and/or a component of an atherogenic lipoprotein to determine that the proteosome inhibitor does not substantially impair the efficacy of the compound, more particularly, the efficacy of stimulating PERPP. Possible proteosome inhibitors include, but are not limited to, lactacystin and ALLN.

The assay of determining that heparin does not substantially impair the efficacy of the compound includes the steps of incubating the hepatic cells with about 10 mg/ml heparin/medium and the compound, measuring the cellular content and/or secretion levels of apoB, an apoB-containing lipoprotein, and/or a component of an atherogenic lipoprotein to determine that heparin does not substantially impair the efficacy of the compound, more particularly, the efficacy of stimulating PERPP.

The assay of determining that the efficacy of the compound is substantially reversible by an antioxidant includes the steps of incubating the hepatic cells with the antioxidant and the compound, measuring the cellular content and/or secretion levels of apoB, an apoB-containing lipoprotein, and/or a component of an atherogenic lipoprotein to determine that the efficacy of the compound is substantially reduced or inhibited by the presence of the antioxidant. Possible antioxidants include, but are not limited to, vitamin E, desferroxammine (DFX), butylhydroxytoluene (BHT), and EGTA.

Similar steps also may be used in determining that the efficacy of the compound is substantially reversible by a PI-3 kinase inhibitor. Possible PI-3 kinase inhibitors include, but are not limited to, wortmannin, LY compound (290004), anti-sense inhibitors, RNA interference inhibitors, and ribozyme inhibitors.

The second assay also includes, but is not limited to, 1) determining that the compound does not substantially affect apoB level in the ER; and 6) determining that the compound substantially increases cellular content of reactive oxygen species (ROS) measured by TBARS assay.

The present invention also provides methods of screening for at least one gene, in which the at least one gene or the RNA(s) or protein(s) encoded thereof regulate intracellular apoB degradation through PERPP. First, genetically engineered hepatic cells containing a mutation that affects the at least one gene may be constructed. Further steps include, measuring cellular content and/or secretion levels of apoB, an apoB-containing lipoprotein, and/or a component of an atherogenic lipoprotein from the genetically engineered hepatic cells to determine that the at least one gene or the RNA(s) or protein(s) encoded thereof regulate the intracellular apoB degradation in combination with at least one second assay. The second assay includes, but is not limited to, the steps of 1) determining that a proteosome inhibitor does not substantially impair the regulation of intracellular apoB degradation by the at least one gene or the RNA(s) or protein(s) encoded thereof; 2) determining that heparin does not substantially impair the regulation of intracellular apoB degradation by the at least one gene or the RNA(s) or protein(s) encoded thereof; 3) determining that the genetically engineered hepatic cells exhibit substantially elevated or reduced apoB level in Golgi, but not ER; and 4) determining that the genetically engineered hepatic cells exhibit substantially elevated or reduced cellular content of ROS.

Genetically engineered hepatic cells containing a mutation may be prepared by a number of methodologies that are well known to those skilled in the art. Literature sources for genetic engineering include Berger & Kimmel (Guide to Molecular Coning Techniques, Methods in Enzyinology, Vol. 152, Academic Press, Inc., San Diego, Calif.); Sambrook et al. (Molecular Cloning—A Laboratory Manual, 2d Edition, Vol. 1-3, Cold Spring Harbor Laboratory, Cold Spring Harbor Press, New York, 1989); and Current Protocols in Molecular Biology (Ausubel et al., Eds., Current Protocols, 1994 Supplement). Useful information also is found in product information from manufacturers of biological regents and experimental equipment, such as the SIGMA Chemical Company (St. Louis, Miss.), R&D Systems (Minneapolis, Minn.), Pharmacia LKB Biotechnology (Piscataway, N.J.), CLONTECH Laboratories, Inc. (Pal Alto, Calif.), Chem Genes Corp., Aldrich Chemical Company(Milwaukee, Wis.), Fluka Chemica Biochemika Analytika (Fluka Chemie AG, Buchs, Switzerland), and Applied Biosystems (Foster City, Calif.). Methods for the polymerase chain reaction (PCR), the ligase chain reaction (LCR), Q.β.-replicase amplification and other RNA polymerase mediated techniques (e.g., NASBA) are also Mullis et al. (U.S. Pat. No. 4,683,202); PCR Protocols A Guide to Methods and Applications (Innis et al., Eds., Academic Press Inc., San Diego, Calif., 1990), Arnheim et al., (C&EN, 36-47, Oct. 1, 1990); The Journal Of NIH Research (3: 81-94, 1991); Kwoh et al. (Proc. Natl. Acad. Sci. USA, 86: 1173, 1989); Guatelli et al. (Proc. Natl. Acad. Sci. USA, 87: 1874, 1990); Lomell et al. (J. Clin. Chem, 35: 1826, 1989); Landegren et al. (Science 241: 1077-1080, 1988); Van Brunt (Biotechnology 8: 291-294, 1990); Wu & Wallace (Gene 4: 560, 1989); Barringer et al. (Gene 89: 117, 1990); and Sooknanan & Malek (Biotechnology 13: 563-564, 1995).

The gene(s) or the RNA(s) or protein(s) encoded thereof, identified by this methods may be used as genetic markers in diagnosing a cardiovascular or metabolic disorder or syndrome that is associated with high plasma levels of apoB, apoB-containing lipoproteins, or atherogenic lipoproteins. The gene(s) and the RNA(s) or protein(s) encoded therefrom also may be used as target(s) or lead compound(s) for drug screening.

The present invention also provides methods of treating a patient by administering a therapeutically effective amount of a Therapeutic that stimulates PERPP to reduce plasma concentration of apoB, apoB-containing lipoproteins, and/or components of an atherogenic lipoprotein.

The patient is preferably an animal, including but not limited to animals such as cows, pigs, chickens, etc., and is preferably a mammal, and most preferably human. The patient may have a cardiovascular disorder and/or a hyperlipidemia, including but not limited to, angina, atherosclerosis, restenosis, claudication, unstable angina, stroke, transient ischemic attacks, coronary artery disease, peripheral vascular disease, cerebral vascular disease, endothelial dysfunction, elevated plasma concentration of a LDL, elevated plasma concentration of a VLDL, elevated plasma concentration of a lipoprotein(s), elevated plasma concentration of an apoB-containing lipoprotein, elevated plasma concentration of a beta-VLDL, elevated plasma concentration of an atherogenic lipoprotein, and a syndrome recommended for treatment by the Adult Treatment Panel of the National Cholesterol Education Program (see, for example, JAMA 285:2486-2497, May 16, 2001); or the patient may have a metabolic disorder or syndrome, including but not limited to, hypercholesterolemia, hyperlipidemia, hyper lipoproteinemia, familial hyperlipidemia, familial combined hyperlipidemia, syndrome X, insulin resistance syndromes, diabetes, secondary hyperlipidemia, secondary insulin resistance, hyperlipidemia and/or insulin resistance that is secondary to a medication, hyperlipidemia and/or insulin resistance that is secondary to an anti-HIV medication, and metabolic disorders associated with vascular disease.

Potential Therapeutics that stimulate PERPP include, but are not limited to, a PI-3 kinase inducer, poly-unsaturated fatty acids, a Therapeutic that lowers hepatic content of thioredoxin, a Therapeutic that lowers hepatic content of vitamin E, a Therapeutic that inhibits transfer of vitamin E, a Therapeutic that inhibits transfer of vitamin E by a phospholipid transfer protein, and a Therapeutic that induces ROS.

In a preferred aspect, the Therapeutic is substantially purified.

Various delivery systems are known and used to administer a Therapeutic of the invention, e.g., encapsulation in liposomes, microparticles, microcapsules, expression by recombinant cells, receptor-mediated endocytosis (see, e.g., Wu and Wu, 1987, J Biol Chem, 262:4429-4432), etc. Methods of introduction include but are not limited to intradermal, intramuscular, intraperitoneal, intravenous, subcutaneous, intranasal, and oral routes. The compounds are administered by any convenient route, for example by infusion or bolus injection, by absorption through epithelial or mucocutaneous linings (e.g., oral mucosa, rectal and intestinal mucosa, etc.) and are administered together with other biologically active agents.

In a specific embodiment where the Therapeutic is a nucleic acid encoding a protein therapeutic, the nucleic acid is administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot, et al., 1991, Proc Natl Acad Sci USA, 88:1864-1868), etc. Alternatively, a nucleic acid therapeutic is introduced intracellularly and incorporated within host cell DNA for expression, by homologous recombination.

The present invention also provides pharmaceutical compositions. Such compositions comprise a therapeutically effective amount of a Therapeutic that induces PERPP, and a pharmaceutically acceptable carrier or excipient. Such a carrier includes but is not limited to saline, buffered saline, dextrose, water, glycerol, ethanol, and combinations thereof. The carrier and composition are sterile. The formulation should suit the mode of administration.

The composition, if desired, can also contain minor amounts of wetting or emulsifying agents, or pH buffering agents. The composition is a liquid solution, suspension, emulsion, tablet, pill, capsule, sustained release formulation, or powder. The composition is formulated as a suppository, with traditional binders and carriers such as triglycerides. Oral formulation includes standard carriers such as pharmaceutical grades of mannitol, lactose, starch, magnesium stearate, sodium saccharine, cellulose, magnesium carbonate, etc.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it is dispensed with an infusion bottle containing sterile pharmaceutical grade water or saline. Where the composition is administered by injection, an ampoule of sterile water for injection or saline is provided so that the ingredients are mixed prior to administration.

The Therapeutics that stimulate PERPP of the invention are formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The amount of the Therapeutic of the invention which will be effective in the treatment of a particular disorder or condition will depend on the nature of the disorder or condition, and can be determined by standard clinical techniques. In addition, in vitro assays are employed to help identify optimal dosage ranges. The precise dose to be employed in the formulation will also depend on the route of administration, and the seriousness of the disease or disorder, and should be decided according to the judgment of the practitioner and each patient's circumstances. Effective doses are extrapolated from dose-response curves derived from in vitro or animal model test systems.

The invention also provides a pharmaceutical pack or kit comprising one or more containers filled with one or more of the ingredients of the pharmaceutical compositions of the invention. Optionally associated with such container(s) is a notice in the form prescribed by a governmental agency regulating the manufacture, use or sale of pharmaceuticals or biological products, which notice reflects approval by the agency of manufacture, use or sale for human administration.

Still within the scope of the present invention are methods of treating subjects by administering a Therapeutic that stimulates PERPP in combination with another type of cholesterol lowering drug. These other types of cholesterol lowering drugs include, but are not limited to, HMG CoA reductase inhibitors, squalene synthetase inhibitors, fibric acid derivatives, probucols, bile acid sequestrants, nicotinic acids and neomycins. An HMG CoA reductase inhibitor includes, but is not limited to, pravastatin, lovastatin, simvastatin, atorvastatin, fluvastatin and cerivastatin. A fibric acid derivative includes, but is not limited to, gemfibrolzil, fenofibrate, clofibrate, bezafibrate, ciprofibrate, and clinofibrate. Other agents include, but are not limited to, dextrothyroxine or its sodium salt, colestipol or its hydrochloride, cholestyramine, nicotinic acid, neomycin, p-aminoaslicylic acid or aspirin. Representative lipid-lowering drugs can be found in The Medical Letter on Drugs and Therapeutics, vol. 43, issue 1105, pp. 43-48, May 28, 2001, which is incorporated herein by reference.

Highly active anti-retroviral therapies, which incorporate HIV protease inhibitors, ameliorate many AIDS related illnesses. Methods of screening for HIV protease inhibitors are subjects of a number of U.S. patents. For example, U.S. Pat. No. 5,252,477 discloses the sequence for HIV protease, and U.S. Pat. No. 5,171,662 provides methods of identifying HIV protease inhibitors (incorporated herein by reference).

However, patients receiving protease inhibitors often develop a marked lipodystrophy and hyperlipidemia. As demonstrated by the present invention, under physiologically relevant conditions, the ERAD is close to zero, and the basal degradation of apoB under these conditions mainly is mediated by PERPP. Therefore, the hyperlipidemia caused by HIV protease inhibitors in vitro is mainly due to the inhibition of PERPP. Thus, another embodiment of the present invention, is to provide methods of identifying an HIV protease inhibitor that exhibits reduced or absent inhibition of intracellular apB degradation through PERPP, which methods include the steps of contacting hepatic cells which exhibit PERPP with the HIV proteasae inhibitor; and 2) measuring cellular content and/or secretion levels of apoB, an apoB-containing lipoprotein, and/or a component of an atherogenic lipoprotein to determine the reduced or absent inhibition of intracellular apoB degradation by the HIV protease inhibitor. To maximize the PERPP pathway in cultured primary mammalian hepatic cells, cells may be plated in a rich medium, such as, about 10% FBS and about 10% horse serum, for a total of 20% serum, volume/volume, for at least about 48 hours prior to the experiment.

Also within the scope of the present invention are methods of treating an HIV-infected individual by administering a therapeutically effective amount of an HIV protease inhibitor, which exhibits reduced or absent inhibition of intracelluar apoB degradation. Still within the scope of the present invention, are methods of treating an HIV-infected individual by administering an HIV protease inhibitor in combination with a compound that stimulates PERPP.

Methods

Male Sprague-Dawley rats (Ace Animals, Boyertown, Pa.) weighing 200-225 g were used to obtain hepatocytes by a protocol approved by the institutional animal care committee. All reagents, unless otherwise specified, were purchased from Sigma (St. Louis, Mo.). [¹⁴C]eicosapentaenoic acid, [³⁵S] methionine, and [14C]triolein, and Enhance solution were purchased from New England Nuclear (Boston, Mass.); [¹⁴C]oleic acid and 1-palmitoyl-2-[¹⁴C]oleoyl phosphatidyl choline (POPC) were purchased from Amersham (Arlington Heights, Ill.). Immunoprecipitin (staph A cells) was purchased from Bethesda Research Labs (Gaithersburg, Md.). Collagenase was purchased from Worthington Biochemical (Freehold, N.J.). Rat hepatoma (McArdle RH-7777) and human hepatocarcinoma (HepG2) cells were purchased from American Type Tissue Collection (Manassas, Va.). Rabbit polyclonal antisera to rat apoB or apoE and mouse monoclonal antibody to rat apoB were developed in the laboratories and were previously described in Wang, H., Chen, X., and Fisher, E. A. (1 993) J Clin Invest 91(4), 1380-9; Sparks, J. D., Bolognino, M., Trax, P. A., and Sparks, C. E. (1986) Atherosclerosis 61(3), 205-11; Sparks, J. D., Zolfaghari, R., Sparks, C. E., Smith, H. C., and Fisher, E. A. (1992) J Clin Invest 89(5), 1418-30.

Cell Culture Techniques

Rats fed ad-libitum were sacrificed in the morning, and liver cells were isolated by collagenase perfusion using 0.225 mg collagenase/ml dissolved in Krebs-Ringer buffer containing 1.66 mM calcium. Hepatocytes were purified by differential centrifugation through a 45% Percoll solution, and their viability was determined by exclusion of ethidium bromide stain, using a fluorescence microscope to visualize the stained nuclei of damaged cells. Only preparations with >90% intact cells were used.

Cells were plated at a density of 2×10⁶ cells/ml on 60-mm culture dishes (previously coated with poly-D-lysine) in modified M199 medium (M199, 1% fetal bovine serum [FBS], 1 mM nicotinamide, 0.1 nM insulin, 3 mg/ml choline, 1.1% L-glutamine, 1% BSA). After a four-hour attachment period, the medium was changed (modified M199 identical to the above except BSA was omitted and the concentration of FBS was raised to 10%). The next morning, cells were washed in serum-free medium three times, the experimental media added, and the cells incubated at 37° C. for 4-6 hours. The experimental medium consisted of serum-free RPMI containing the appropriate isotopes (see below) and fatty acids (FA) present at a final concentration of 0.8 mM complexed to BSA (FA:BSA molar ratio=5:1). The fatty acids used were oleic (OA), EPA, and DHA. Control medium was identical, except that BSA (0.16 mM) without fatty acids was present. For metabolic labeling of proteins, [³⁵S]methionine (70 uCi/ml) was included in the experimental media.

As indicated in Results, in some experiments, McArdle RH-7777 (rat hepatoma) or HepG2 (human hepatocarcinoma) cells, maintained as in Williams, K. J., Brocia, R. W., and Fisher, E. A. (1990) J Biol Chem 265, 16741-16744; Wang, H., Yao, Z., and Fisher, E. A. (1994) J Biol Chem 269(28), 18514-20, were substituted for the rat primary hepatocytes.

Secretion of Newly Synthesized Apoproteins

Following the 4-6 hour incubation period, the cell monolayer was washed, then solubilized in 0.1 M NaOH, and the cell protein determined by the Lowry method (Lowry, O. H., Rosebrough, N. J., Farr, A. L., and Randall, R. J. (1951) J Biol Chem 193, 265-75). Each 60 mm dish contained approximately 1-1.5 mg of cell protein. The secreted apoproteins were isolated from the medium either by ultracentrifugation or immunoprecipitation. To isolate the d<1.006 (VLDL) and 1.006<d<1.063 (IDL and LDL) g/ml density classes by centrifugation, sequential 20 hour runs at 48,000 rpm in a 50 Ti Beckman rotor at 4° C. were performed. After the first run at d=1.006, the upper VLDL layer was harvested and the density of the infranatant adjusted to 1.066 g/ml with solid KBr. After being overlaid with a KBr solution of density 1.063, the adjusted infranatant was re-centrifuged as before to isolate the IDL and LDL fractions. In some experiments, the HDL fraction (1.063<d<1.21) was isolated by sequential density centrifugation. Lipoprotein fractions were then dialyzed against 0.9% NaCl containing 10 mM unlabeled methionine, 0.2 mM PMSF, and 2 mM EDTA. Dialyzed lipoproteins were delipidated using 9 volumes of 100% isopropanol. Apoproteins were then collected by centrifugation in a Sorval HB-4 rotor at 10,000 rpm and 4° C. for 20 min, and dissolved in buffer (0.01 M phosphate, pH 7,1 % SDS, 10% glycerol), in preparation for electrophoresis.

To immunoprecipitate apoB or apoE from unfractionated conditioned medium, monospecific rabbit antiserum to rat apoB or apoE was used. Briefly, an aliquot of the medium was mixed with an equal volume of diluted antiserum (1:100 in PBS and 1% BSA) and incubated overnight at 4° C. Staph A was added in the form of immunoprecipitin, and the resultant precipitate containing the immune complexes was washed extensively, the staph A cells removed, and the isolated apoprotein dissolved in gel sample buffer (0.0625M tris/Cl, pH 6.8, 20% glycerol, 2% SDS). Electrophoretic separation of apoVLDL, apoIDL/LDL, and immunoprecipitates was accomplished using 3.5% acrylamide-18% glycerol gels as described (Maguire, G. F., Lee, M., and Connelly, P. W. (1989) J Lipid Res 30, 757-61). An aliquot of each sample was taken for scintillation counting, so that total radioactivity applied to each lane could be determined. Also, protein size standards were included in each gel.

After electrophoresis, the gels were stained, fixed, soaked in Enhance fluor solution, and dried following protocols supplied by the manufacturer. Dried gels were then exposed to X-ray film at −70° C. and signals on the resulting fluorograms were typically quantified by densitometry or phosphorimaging. Using the total radioactivity applied to each lane of the gel and the relative signal intensity of each band in that lane, the radioactivity associated with each apoprotein species was calculated. In some cases, gel bands were excised and the incorporated radioactivity measured directly by scintillation counting. As a control, the incorporation of [³⁵S]methionine into total cellular and secreted proteins was measured by TCA-PTA precipitation, as described in Wang, H., Chen, X., and Fisher, E. A. (1993) J Clin Invest 91(4), 1380-9.

To determine the differential effects of MTP inhibition on the secretion of labeled apoB and apoE, in some experiments, McArdle RH-7777 cells were pre-treated for 30 min with MTP inhibitor BMS-200150 (10 mM dissolved in DMSO; (Jamil, H., Gordon, D. A., Eustice, D. C., Brooks, C. M., Dickson, J. K., Jr., Chen, Y., Ricci, B., Chu, C.-H., Harrity, T. W., Ciosek, C. P., Jr., Biller, S. A., Gregg, R. E., and Wetterau, J. R. (1996) Proc Natl Acad Sci USA 93, 11991-11995)) or DMSO alone (final concentration 0.5%) before adding radiolabel. The cells were further incubated for 4 hr and the conditioned media contents of apoB and apoE determined by immunoprecipitation and SDS-PAGE analysis as described above.

To determine the effects of inhibiting PI3-kinase on apoB secretion, rat primary hepatocytes were pre-treated for 30 min with 1 μM wortmannin (dissolved in DMSO) or DMSO alone (final concentration 0.5%), which were maintained throughout the experiment. DHA/BSA complexes were then added and the cells were further incubated for 4 hr. The apoB contents of the conditioned media samples were determined by radioimmunoassay (Sparks, J. D., Bolognino, M., Trax, P. A., and Sparks, C. E. (1986) Atherosclerosis 61(3), 205-11). In some experiments, [³⁵S]methionine was used to pulse label apoB. The effect of wortmannin on labeled apoB recovery from cell lysate and conditioned medium at the 15 min and 90 min time points of the chase period was determined by immunoprecipitation and SDS-PAGE analysis (Wang, H., Chen, X., and Fisher, E. A. (1993) J Clin Invest 91(4), 1380-9).

In some experiments, the protein trafficking inhibitor Brefeldin A (BFA) was used to assess whether Ω-3 fatty acids affected event(s) post-ER. Rat hepatoma cells were incubated at 37° C. for 4 hr in [³⁵S]methionine-containing medium supplemented with either DHA complexed to BSA (DHA/BSA) or DHA/BSA plus 4 mg/ml BFA. The total recovery of labeled apob,₀₀ was determined by immunoprecipitation of cell lysates and media samples, followed by SDS-PAGE and phosphorimager analysis.

To determine whether the proteasome mediated Ω-3 fatty acid-induced apoB degradation, rat hepatoma cells were incubated at 37° C. for 4 hr in [³⁵S]methionine-containing medium supplemented with either BSA or EPA/BSA complexes in the absence or presence of the proteasome inhibitor, lactacystin (10 mM; purchased from the laboratory of Dr. E. J. Corey, Harvard University). Samples of cell lysates and conditioned media were subjected to immunoprecipitation analysis with anti-apoB antiserum, followed by SDS-PAGE and fluorography. To determine whether Ω-3 fatty acids targeted apoB to lysosomal degradation, a similar experiment was performed substituting ammonium chloride (40 mM) for lactacystin. At this concentration of ammonium chloride, there was no evidence of cell toxicity as assessed by TCA-PTA precipitation analysis, but there was >80% inhibition of the degradation of exogenously added ¹²⁵I-LDL (a generous gift of Dr. Ira Tabas, Columbia University).

Effects on Nascent VLDL Re-Uptake

To evaluate whether there were differential effects of fish oil fatty acids and OA on the re-uptake of newly synthesized VLDL, an experimental design identical to that described above was employed, except heparin (0.1 mg/ml or 10 mg/ml) was added to the treatment media to block LDL receptor-dependent and proteoglycan-mediated re-uptake (Williams, K. J., Fless, G. M., Petrie, K. A., Snyder, M. L., Brocia, R. W., and Swenson, T. L. (1992) J Biol Chem 267(19), 13284-92). Control experiments were performed showing that the flotation properties of VLDL were unchanged in the presence of heparin.

Effects on Microsomal Triglyceride Transfer Protein Activity

Three methods were used to assess possible effects of Ω-3 fatty acids on cellular MTP function. First, rat primary hepatocytes were incubated with BSA, OA, or EPA, and then MTP activity in cell lysates was measured using a fluorescent assay (Roar Biomedical, New York, NY). Second, to determine if lipid classes synthesized in the presence of OA or Ω-3 fatty acid are equivalent substrates for MTP, primary rat hepatocytes or HepG2 cells were incubated with tritiated OA or EPA, total cellular lipids were extracted into isopropanol, and biosynthetically labeled cholesteryl ester, triglyceride, and phospholipids were separated by preparative thin layer chromatography. Each of these isolated lipids was incorporated into vesicles that were used as donors in a transfer reaction with acceptor vesicles and 50 pg of purified bovine MTP, as described in Jamil, H., Dickson, J. K., Jr., Chu, C. H., Lago, M. W., Rinehart, J. K., Biller, S. A., Gregg, R. E., and Wetterau, J. R. (1995) J Biol Chem 270(12), 6549-54. As an internal control for lipid transfer, each donor vesicle also contained [¹⁴C]triolein. Third, to determine if Ω-3 lipids inhibit MTP-mediated transfer of non-Ω-3 lipids, artificial donor vesicles were prepared containing either labeled triolein or labeled POPC. The majority of lipid mass in these vesicles was unlabeled phosphatidylcholine that contained either 100% oleate esterified at the sn-2 position (control) or a mixture of 70% oleate and 30% DHA or 30% EPA. Transfer of the labeled lipids to acceptor vesicles in the presence of 50 μg of purified bovine MTP was measured as described in Jamil, H. et al (1995) J Biol Chem 270(12), 6549-54.

Sub-Cellular Fractionation of Rat Primary Hepatocytes

Rat primary hepatocytes were isolated as above, allowed to recover overnight, and then incubated for 4 hr in DMEM supplemented with [³⁵S]methionine (300 μCi/ml), 0.16 mM BSA, and either no fatty acids, 0.8 mM OA, or 0.8 mM DHA. The monolayers were washed three times, and the cell lysates and post-nuclear supernatants were prepared as described in Furukawa, S. et al (1992) J Biol Chem 267(31), 22630-8. The cell lysates were mixed with sucrose to a final concentration of 8.58%, then placed onto discontinuous sucrose gradients. The density layers were: 56% sucrose (0.46 ml), 50% (0.92 ml), 45% (1.38 ml), 40% (2.3 ml), 35% (2.3 ml), 30% (1.38 ml), 20% (0.46 ml) and 8.58% (2.3 ml of the post-nuclear supernatant). After ultracentrifugation (SW41 rotor, 4° C., 39,000 rpm, 18 hr), 23 fractions of 0.5 ml each were collected from the top of the tube. From each of the top 22 fractions, 420 μl was subjected to immunopreciptation/SDS-PAGE analysis using rabbit anti-rat apoB antiserum, followed by fluorography then densitometric quantification; 4 μl was used to assay the Golgi marker enzyme α-mannosidase II (Storrie, B. et al (1990) Methods Enzymol 182, 203-25); and 11 μl was used for Western blot analysis of the ER membrane protein calnexin (using an antibody from Calbiochem) followed by densitometric quantification by densitometry.

Statistical Analysis

Results are displayed as mean ±SE, n≧3. Absent error bars in figures indicate SE values smaller than the drawn symbols. For comparisons between a single experimental group and a control, the unpaired, two-tailed t-test was used. For comparisons involving several groups simultaneously, analysis of variance (ANOVA) was initially used. When the ANOVA indicated differences amongst the groups, pairwise comparisons of each experimental group versus the control group were performed using the Bonferroni test or Dunnett q′ statistic. Analyses were performed with the PRISM (GraphPAD Software, San Diego, Calif.).

Results

Nature of the Target for Ω-3 Fatty Acid-Induced Degradation:

Prior studies using rat hepatoma McArdle RH7777 (McA) cell clones that express a range of artificially truncated human apoB constructs have suggested that buoyant lipoproteins, regardless of the precise length of the transfected apoB construct, are the most susceptible to degradation induced by Ω-3 fatty acids (Wang, H. et al. (1993) J Clin Invest 91(4), 1380-9; Wang, H. et al. (1994) J Biol Chem 269(28), 18514-20). Two approaches were used to test this suggestion with native, rather than artificially truncated, forms of apoB. First, rat primary hepatocytes were studied. This cell type is a non-transformed cell that secretes apoB₁₀₀ almost exclusively in the form of VLDL, while dividing its production of apoB₄₈ between particles with the density of VLDL (˜⅔ of secreted apoB₄₈) and HDL (˜⅓ of secreted apoB₄₈) (Hussain, M. et al. (1989) Biochim Biophys Acta 1001 (1), 90-10134).

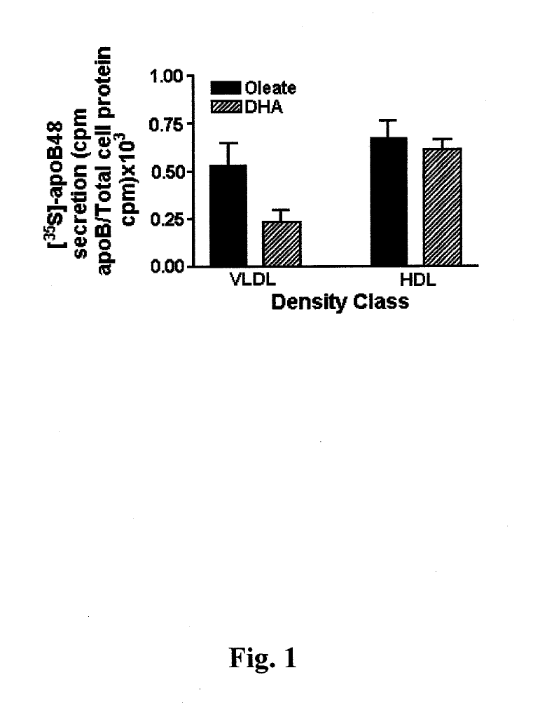

Rat primary hepatocytes were incubated at 37° C. for 4 hr with OA or DHA complexed to BSA (or BSA alone) in the presence of [³⁵S]methionine, and the conditioned media were separated into VLDL and HDL fractions by density gradient ultracentrifugation. The content of labeled apoB₄₈ in each density fraction was then quantified. As shown in FIG. 1, the recovery of labeled apoB₄₈ from the VLDL fraction of conditioned media from cells treated with DHA averaged only ˜35% (P<0.001) compared to the results with samples from the OA group, whereas the recovery of labeled apoB₄₈ from the HDL fraction was essentially independent of treatment. Thus, a single type of apoB, apoB₄₈, was differentially affected by DHA depending entirely on the density of the associated lipoprotein.

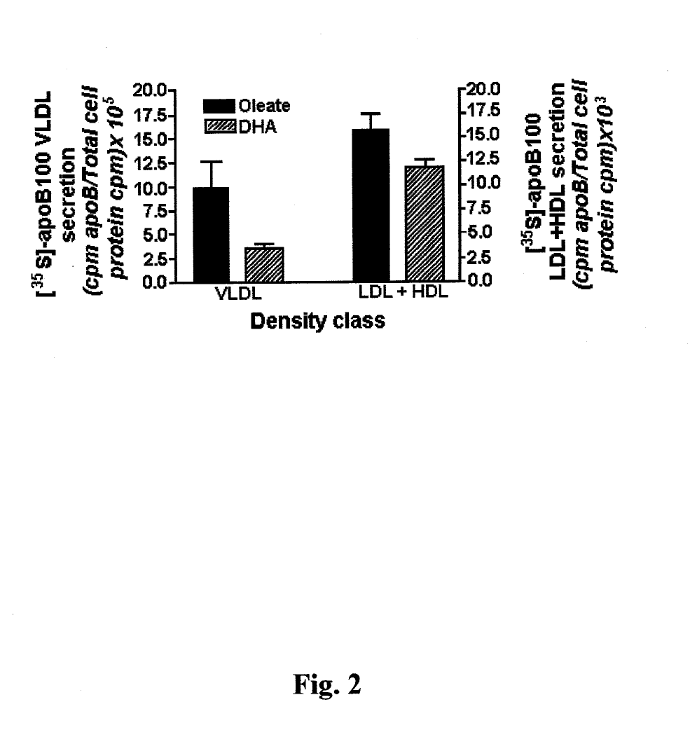

To pursue the implication that the apoB sequence per se is not a determinant of Ω-3 fatty acid-induced degradation, a different cell culture model was utilized, the human hepatocarcinoma HepG2, which produces apoB₁₀₀, but no apoB₄₈. A small, but easily measurable amount of HepG2 apoB₁₀₀ is secreted as part of lipoproteins with the density of VLDL, with the majority appearing in the denser IDL/LDL (1.006<d<1.063 g/ml) and HDL (1.063<d<1.21) fractions. In previous studies (Wong, S. et al. (1989) Arterioscler Thromb 9, 836-841), it was found that EPA or DHA treatment significantly reduced the secretion of newly synthesized VLDL-apoB₁₀₀ by HepG2 cells, but the effects on the denser apoB-lipoproteins were not examined. Therefore, HepG2 cells were incubated 4 hr with [35 S]methionine and either OA or DHA, and the conditioned media were separated by centrifugation into fractions of d<1.006 g/ml (VLDL) and 1.006<d<1.21 g/ml (LDL+HDL). The recoveries of apoB₁₀₀ from each density fraction are summarized in FIG. 2.

As expected, a small amount of labeled apoB₁₀₀ was secreted into the VLDL fraction in either treatment group (FIG. 2, left 2 bars; note the difference in the scale of the left and right Y-axes). Nevertheless, the relative recovery of newly secreted VLDL-apoB₁₀₀ was considerably lower after DHA treatment (˜35% of that in the OA group; P<0.01, consistent with FIG. 1). In contrast, DHA treatment only mildly affected the relative recovery of labeled apoB₁₀₀ from the higher density LDL+HDL class (˜75% of that in the OA group; right 2 bars). Overall, the separate results from the primary hepatocytes, in which apoB₄₈ appears in different density fractions, and HepG2 cells, in which apoB₁₀₀ also appears in different density fractions, clearly demonstrate that it is a property of the lipoprotein particle, not the primary amino acid sequence of apoB, that is a critical factor in determining susceptibility to degradation induced by Ω-3 fatty acids. Similar results have been obtained in rat hepatoma McA cells clones expressing a range of artificially truncated human apoB constructs (Wang, H. et al. (1994) J Biol Chem 269(28), 18514-20). These previous and current results imply a robust phenomenon independent of the primary sequence of apoB.

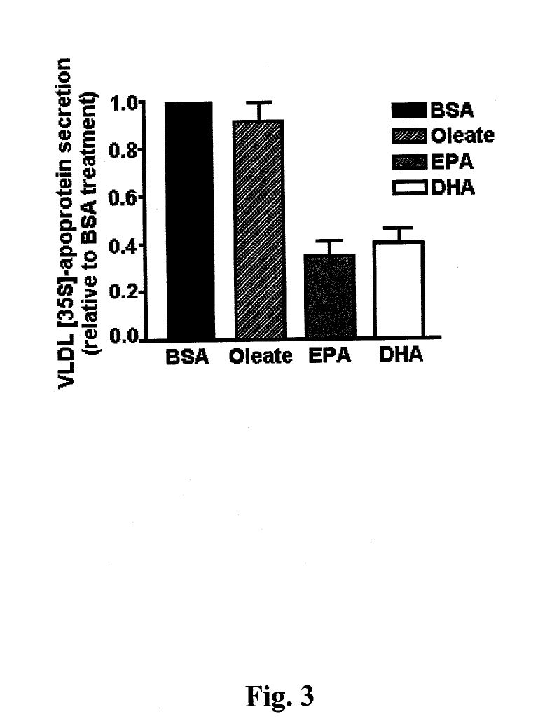

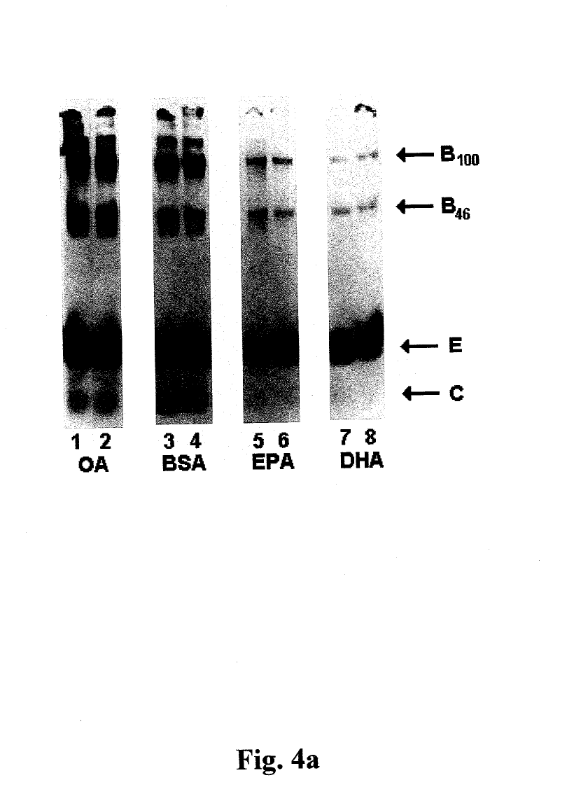

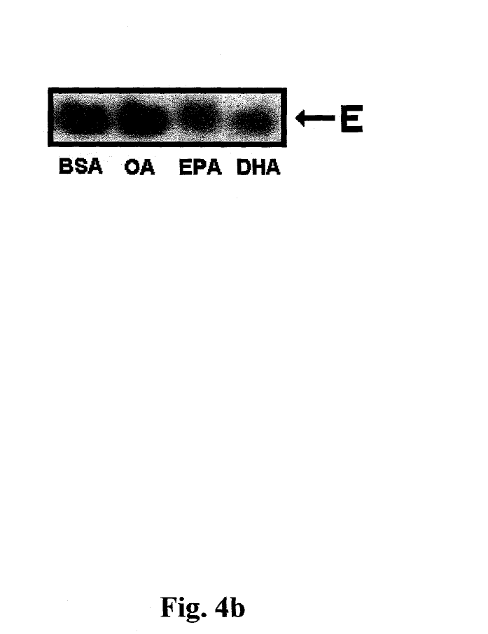

There are two possible explanations for the preferential loss of large, buoyant apoB-lipoproteins in the presence of Ω-3 fatty acids: either Ω-3 fatty acids prevent these particles from being assembled but without allowing the unused apoB to be secreted as higher-density particles, or Ω-3 fatty acids permit the buoyant particles to be assembled, but then selectively induce their destruction. Importantly, these two possibilities would have different consequences for other components of the buoyant particles. When apoB is lost without being assembled into large particles, then the total cellular secretion of other components, particularly apoE, is not affected (e.g., Fazio, S. et al. (1992) Journal of Biological Chemistry 267, 6941-6945; Fazio, S. et al. (1995) Arteriosclerosis and Thrombosis 15, 593-600). In contrast, if entire VLDL particles are removed from the secretory pathway upon Ω-3 fatty acid treatment, then all VLDL apoproteins would be destroyed in parallel. To examine the fate of all of the apoproteins normally associated with VLDL, rat primary hepatocytes were treated with BSA, OA, or Ω-3 fatty acids in the presence of [³⁵S]methionine and then collected in the conditioned media. After centrifugation to isolate the d<1.006 g/ml fraction, samples were delipidated, and the incorporation of |radiolabe into individual species of apoproteins was determined by SDS-PAGE followed by fluorography. As shown in FIG. 3, the secretion of total labeled VLDL-apoproteins was significantly reduced in the Ω-3 fatty acid treated groups relative to the results from the BSA and OA groups (P<0.001). Visual inspection of the fluorograms, such as the one shown in FIG. 4A, indicated that treatment with DHA or EPA produced substantial decreases in the signal intensities of labeled apoB₁₀₀, apoB₄₈, apoE, and apoCs secreted into the medium on VLDL particles. The quantification of the relative signal intensities is summarized in Table 1, except that the results for the apocs were not included, because such a small fraction of radioactivity was attributable to these apoproteins (<5% in any lane), consistent with their being a minor component of VLDL secreted by rat hepatocytes (Hussain, M. et al. (1989) Biochim Biophys Acta 1001(1), 90-101). The results with apoE are particularly informative, because this apoprotein is thought to be added to VLDL particles relatively late in the lipoprotein assembly process (Fazio, S. et al. (1992) Journal of Biological Chemistry 267, 6941-6945; Fazio, S. et al. (1995) Arteriosclerosis and Thrombosis 15, 593-600; and Janero, D. R., and Lane, M. D. (1983) Journal of Biological Chemistry 258, 14496-14504), and until that point, apoE and apoB secretion are independent of each other (Fazio, S. et al. (1992) Journal of Biological Chemistry 267, 6941-6945). Importantly, the results in FIG. 4A can not be explained by a simple re-distribution of apoE to other density classes after administration of Ω-3 fatty acids, because immunoprecipitation of apoE from whole media samples also showed a significant decrease in apoE recovery after EPA or DHA treatment (FIG. 4B). Thus, Ω-3 fatty acids induce a global loss of all VLDL-associated apoproteins, including apoE, consistent with a late effect in the secretory pathway that occurs after considerable assembly of the lipoproteins.

TABLE 1
The secretion of newly synthesized VLDL-apoproteins by rat
hepatocytes incubtaed with fatty acid/BSA complexes, expressed as the
percentage of secretion observed with BSA alone.

| Fatty acid | ApoE | ApoB₄₈ | ApoB₁₀₀ |
|---|---|---|---|
| OA | 84 ± 3 | 132 ± 11 | 96 ± 4 |
| EPA | 45 ± 1 | 45 ± 3 | 25 ± 2 |
| DHA | 53 ± 2 | 39 ± 3 | 24 ± 3 |

Relationship Between Ω-3 Fatty Acid-Induced apoB Degradation and ERAD

ApoB-lipoprotein biogenesis involves an early, regulated degradative process that is mediated by proteasomes (Yeung, S. J. et al. (1996) Biochemistry 35(43), 13843-8; Fisher, E. A. et al. (1997) J Biol Chem 272(33), 20427-34; and Benoist, F. et al. (1997) J Biol Chem 272(33), 20435-42), associated with the endoplasmic reticulum (Mitchell, D. M. et al. (1998) Proc Natl Acad Sci U S A 95(25), 14733-8), and provoked by inadequate MTP-mediated initial lipidation of newly synthesized apoB (Benoist, F. et al. (1997) J Biol Chem 272(33), 20435-42; Zhou, M. et al. (1998) J Biol Chem 273(38), 24649-5332). Although the data point towards events later in the secretory pathway, it was nevertheless necessary to directly examine if Ω-3 fatty acids act at this early step.

First, it was necessary to test the possibility that Ω-3 fatty acids stimulate apob degradation by impeding MTP-dependent early lipidation, thereby targeting apoB to proteasomes. Rat primary hepatocytes were pre-treated for 6 hr with BSA, OA, or EPA, and then the MTP activity in lysates of these cells was assessed using artificial donor and acceptor vesicles, with [¹⁴C]triolein as the MTP substrate, as described in Jamil, H. et al. (1995) J Biol Chem 270(12), 6549-54. No difference in MTP-mediated transfer of labeled triolein was seen in the different cell lysates.

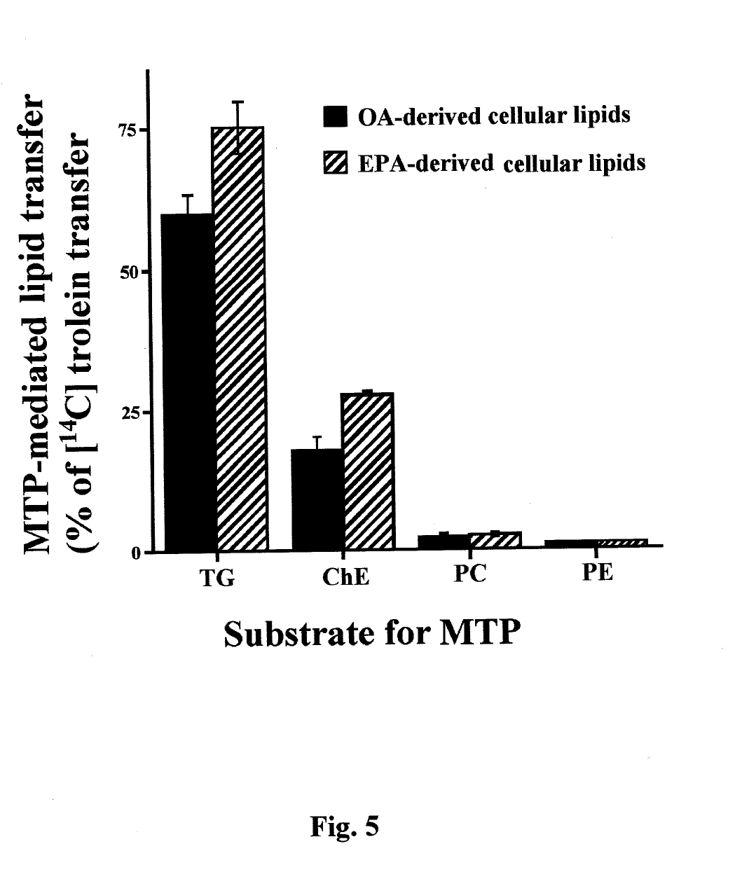

Nonetheless, it is still possible that DHA- or EPA-enriched lipids are poor substrates for MTP-mediated transfer, especially because MTP activity has been reported to exhibit some dependence on the fatty acyl composition of its lipid substrates (Jamil, H. et al. (1995) J Biol Chem 270(12), 6549-54). An intracellular abundance of relatively poor lipid substrates, produced during incubations of hepatocytes with EPA or DHA, could be functionally equivalent to MTP inhibition. Thus, rat primary hepatocytes and HepG2 cells were incubated with radiolabeled OA or EPA to allow incorporation of these fatty acids into lipid esters. After extraction of total cellular lipids, triglycerides, cholesteryl ester, and phospholipids were each isolated by preparative TLC and reconstituted into donor vesicles for a lipid transfer assay, using purified bovine MTP. As shown in FIG. 5, no decrease was seen in MTP-mediated transfer of ³H-labeled triglycerides harvested from EPA-treated cells (rate=75%±10% of [¹⁴C]triolein transfer) versus ³H-triglycerides from OA-treated cells (rate=60%±8% of [¹⁴C]triolein transfer; P=0.3). Likewise, no inhibition was seen for transfer of ³H-cholesteryl esters (EPA: 25%+2% versus OA: 20%+4%; P=0.07), phosphatidylcholines (EPA: 2.6%+0.7% versus OA: 2.3%+1%; P=0.7), or phosphatidylethanolamines (EPA: 1.1%+0.2% versus OA: 1.1%+0.1%; P=0.6). Similar results were also obtained with lipids derived from rat primary hepatocytes treated with labeled OA or EPA for 6 hr.

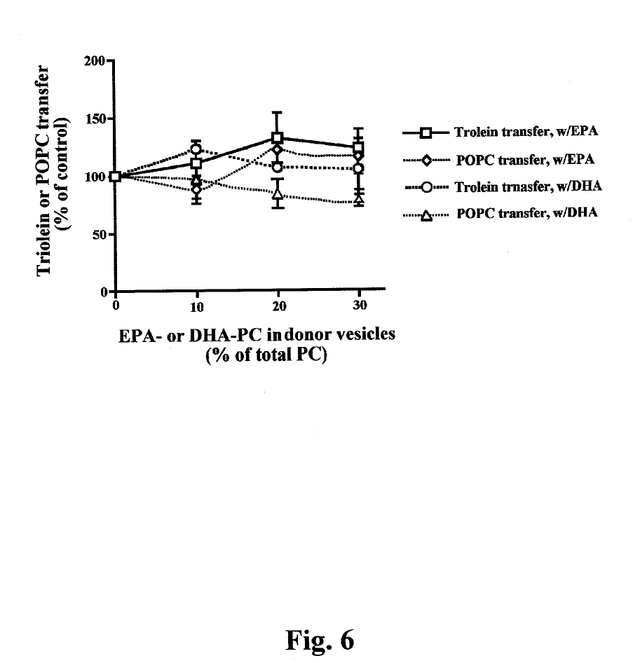

Another approach to detect the effects of Ω-3 fatty acid enrichment on MTP-mediated lipid transfer was to determine if lipids containing Ω-3 fatty acyl groups inhibit the transfer of non-Ω-3 lipids. A totally defined system was prepared, using artificial donor vesicles composed of unlabeled phosphatidylcholine plus [¹⁴C]triolein or 1-palmitoyl-2-[¹⁴C]oleoyl phosphatidylcholine (POPC). As above, the source of MTP was a purified bovine preparation. MTP-mediated transfer of the labeled lipids from these vesicles to an artificial acceptor was then measured. To test the effects of lipids containing Ω-3 fatty acyl groups, different compositions of unlabeled phosphatidylcholine in the donor vesicles were compared, from 0%-30% DHA or EPA groups at the sn-2 position (i.e., 100%-70% oleate esterified at the sn-2 position). As shown in FIG. 6, no significant effects were found on the transfer of labeled triolein or labeled POPC, even when the % enrichment in Ω-3 fatty acids exceeded that achieved in hepatic cells incubated with EPA for 6 hr (Homan, R. et al. (1991) J Lipid Res 32(2), 231-41). Overall, these data do not support the hypothesis that Ω-3 fatty acids interfere with the initial, MTP-dependent phase of VLDL assembly.

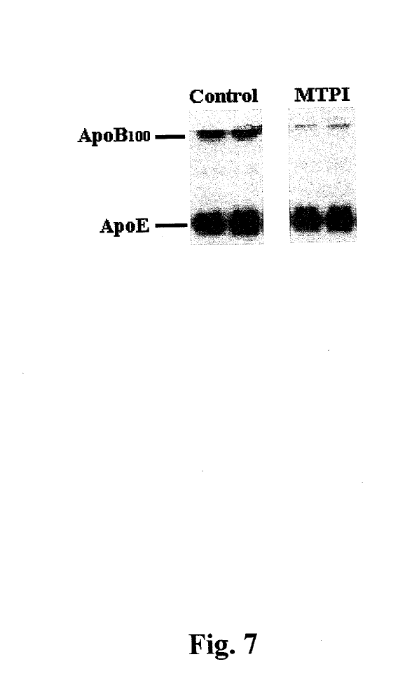

This conclusion is also supported by the pattern of apoprotein secretion after MTP inhibition. Upon treatment of rat hepatoma cells with BMS compound #200150 (Jamil, H. et al. (1996) Proc Natl Acad Sci USA 93, 11991-11995), an inhibitor of MTP, the secretion of newly synthesized apoB and apoE was measured. As seen in FIG. 7, MTP inhibition almost completely abolished the secretion of apoB₁₀₀, as expected (Jamil, H. et al. (1996) Proc Natl Acad Sci USA 93, 11991-11995), but there was no significant effect on apoE secretion. This pattern is unlike the effects of Ω-3 fatty acids, which reduce the secretion of both apoproteins (FIG. 4A and Table 1).

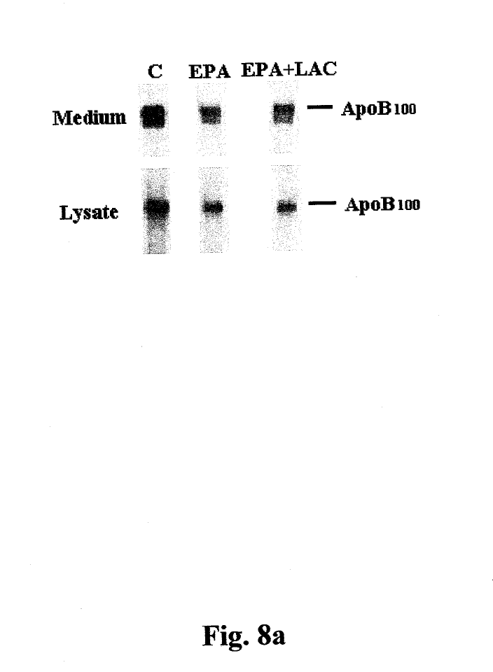

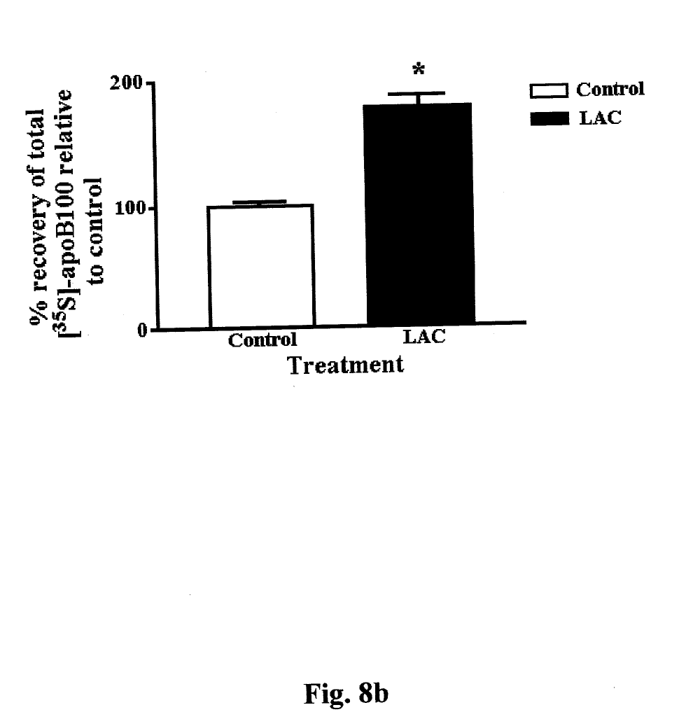

Further proof in living cells that Ω-3 fatty acids affect a step distal to MTP-dependent lipoprotein assembly comes from examining the role of the proteasome, which mediates apoB degradation after inhibition of either lipid synthesis or MTP-mediated lipid transfer (e.g., Mitchell, D. M. et al. (1998) Proc Natl Acad Sci U S A 95(25), 14733-8; Williams, K. J. et al. (1990) J Biol Chem 265, 16741-16744). McA hepatoma cells were treated with either BSA or with EPA/BSA complexes in the absence or presence of the proteasomal inhibitor, lactacystin (Methods). Typical data are shown in FIG. 8, in which lactacystin produced little if any inhibition of EPA-induced degradation (Panel A) at the same time it increased apoB₁₀₀ recovery in the absence of Ω-3 fatty acids (Panel B). Thus, involvement of MTP or ERAD cannot explain four key characteristics of Ω-3 fatty acid-induced degradation: it is specific to buoyant lipoproteins; it occurs without any inhibition of MTP; there is collateral loss of other VLDL apoproteins, particularly apoE; and it is independent from proteasomes.

Relationship Between Ω-3 Fatty Acid-Induced Degradation and Re-Uptake

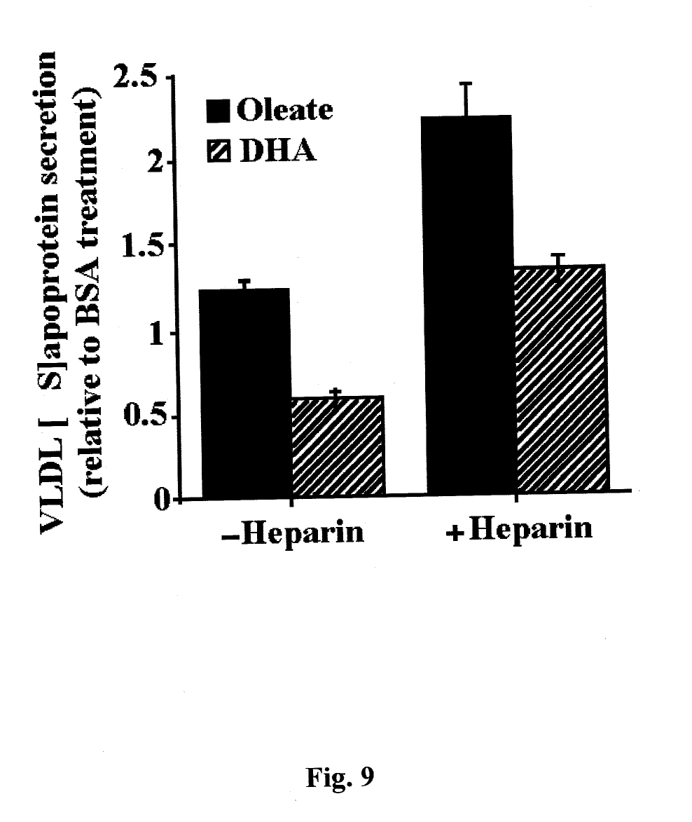

To demonstrate re-uptake of nascent VLDL and to evaluate its potential contribution to the effects of Ω-3 fatty acids, rat primary hepatocytes were treated with either OA or DHA, with or without the addition of heparin to the culture medium. The concentration of heparin was 10 mg/ml, which blocks lipoprotein binding to both LDL receptors and HSPGs (Williams, K. J. et al. (1992) J Biol Chem 267(19), 13284-92, and citations therein). As shown in FIG. 9, the net secretion of newly synthesized VLDL-apoproteins during treatment with either OA or DHA was increased approximately two-fold (P<0.01) by the addition of 10 mg heparin/ml. Thus, there is substantial re-uptake of nascent VLDL by primary hepatocytes in the presence of either fatty acid. Nevertheless, blocking re-uptake with 10 mg heparin/ml did not affect the ability of DHA to reduce VLDL apoprotein output: secretion of VLDL apoproteins in the presence of DHA was approximately 50% of the OA control, independent of heparin treatment. Thus, re-uptake of newly exported apoB cannot explain the effect of Ω-3 fatty acids on lipoprotein secretion.

A similar experiment was conducted with a low concentration of heparin (0.1 mg/ml) that blocks lipoprotein binding to HSPGs without affecting LDL receptor binding (Williams, K. J. et al. (1992) J Biol Chem 267(19), 13284-92). No “bridging molecules,” such as lipoprotein lipase, were added to enhance lipoprotein-HSPG interactions. Under these conditions, the low concentration of heparin failed to increase apoB output in the presence of either fatty acid (cf. FIG. 8 and accompanying text in Williams, K. J. et al. (1992) J Biol Chem 267(19), 13284-92). Thus, under these specific conditions, re-uptake of nascent VLDL is substantial; it is mediated primarily by the binding of apoB₁₀₀ or apoE to the LDL receptor, without significant involvement of cell-surface HSPGs; and the inhibitory effect of Ω-3 fatty acids persists during blockage of re-uptake at the cell surface.

More evidence against the involvement of re-uptake in Ω-3 fatty acid-induced degradation of apoB is based on the knowledge that lipoproteins captured by either LDL receptors (Brown, M. S., and Goldstein, J. L. (1986) Science 232(4746), 34-47) or heparan sulfate proteoglycans (Williams, K. J. et al. (1992) J Biol Chem 267(19), 13284-92; and Fuki, I. V. et al. (2000) J Biol Chem 275(33), 25742-50) are directed to lysosomes. Thus, rat primary hepatocytes cells were treated with either OA or DHA in the absence or presence of ammonium chloride, a lysosomal inhibitor. Under these conditions, ammonium chloride decreased the degradation of ¹²⁵I-LDL, but there was no decrease in DHA-induced degradation of newly synthesized apoB. Overall, involvement of re-uptake cannot explain two key characteristics of Ω-3 fatty acid-induced degradation: it is unaffected by even high concentrations of heparin, which blocks re-uptake of newly exported apoB via LDL receptors and HSPGs, and it is independent from lysosomes.

Intracellular Localization and Signaling Involved in Ω-3-Induced Degradation of apoB

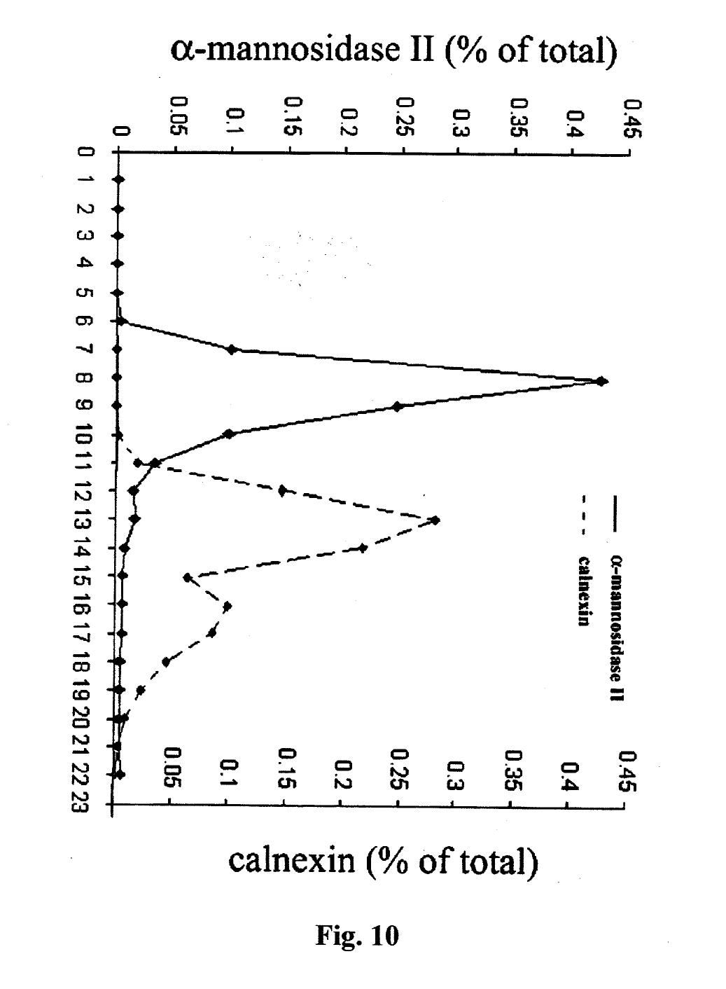

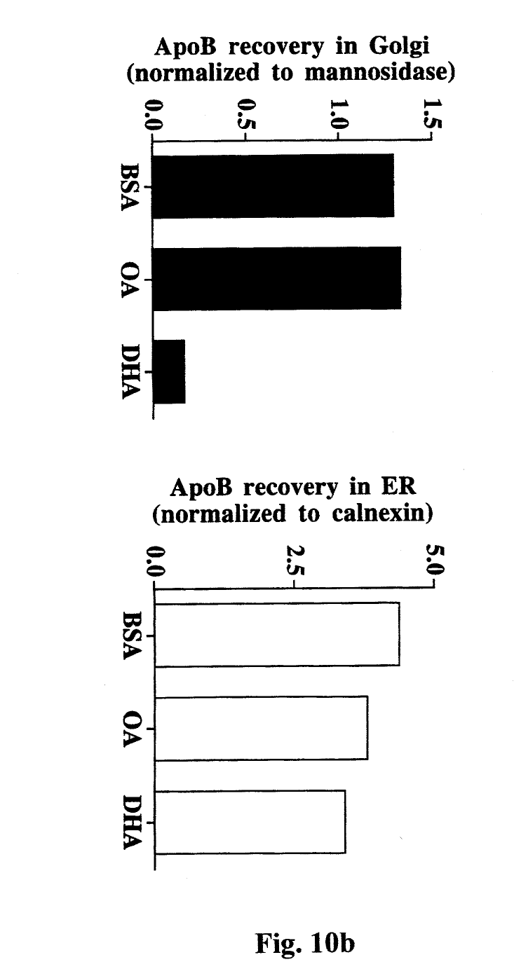

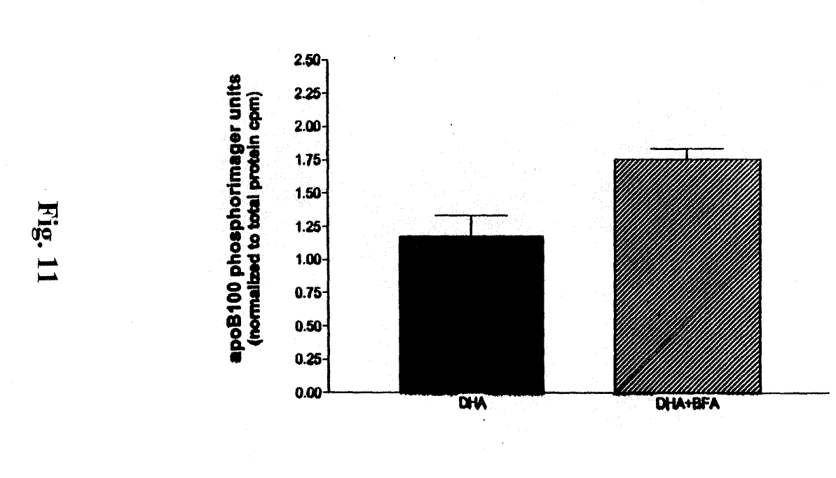

The ERAD-proteasome and cell-surface re-uptake processes represent the initial and the final opportunities, respectively, for a hepatic cell to regulate the net secretion of apoB by targeting it to degradation. Because data disclosed herein do not support a model in which Ω-3 fatty acids exert their effects at either of these steps, it is presumed that they induce a distinct, third ‘threat’ to apoB at an intermediate site that is post-ER but before export across the plasma membrane. To test this possibility, primary rat hepatocytes were subjected to sub-cellular fractionation after treatment with BSA alone or complexes of BSA with either OA or DHA. Based on the distribution of the ER and Golgi markers, calnexin and a-mannosidase II, respectively (FIG. 10, panel A), aliquots were taken from the fractions corresponding to the Golgi (#7-11) and ER (#12-20) and subjected to immunoprecipitation/SDS-PAGE analysis using anti-rat apoB antiserum. ApoB₁₀₀ content was quantified by densitometry, and the sum present in the ER or Golgi fractions was normalized to the recovery of calnexin and a-manosidase II, respectively. As shown in FIG. 10B, the ER-apoB₁₀₀ content was similar among the three groups, whereas treatment with DHA induced a striking loss of apoB₁₀₀ from the Golgi. These results imply that Ω-3 fatty acids induce the degradation of apoB₁₀₀ after it exits the ER. To examine this issue functionally, brefeldin A was used to impede the exit of proteins from the ER. After McA hepatoma cells had been incubated with [³⁵S]methionione for 4 hr in the presence of brefeldin A (4 μg/ml) and DHA, as shown in FIG. 11, the total recovery of labeled apoB₁₀₀ from cell lysates plus medium was 50%+8.3% (n=4) higher than the recovery after treatment with DHA alone, consistent with a post-ER process.

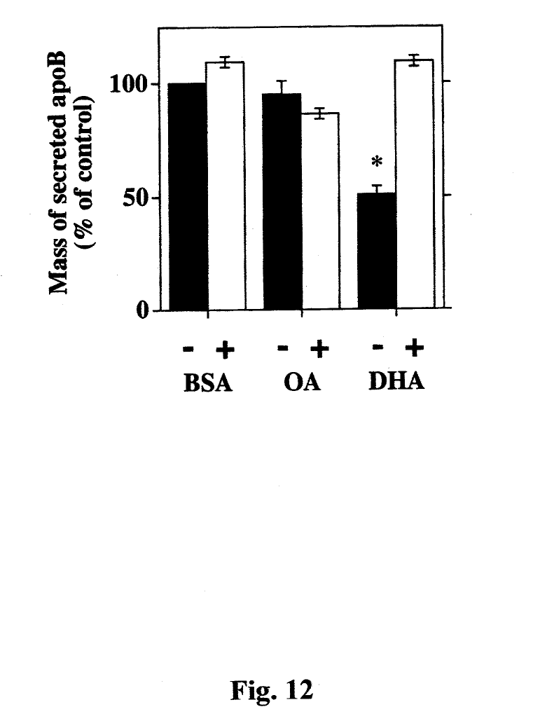

It was recently observed that brefeldin A also protects apoB from acute insulin-stimulated degradation in rat primary hepatocytes (Sparks, J. D. et al. (1996) Biochem J 313(Pt 2), 567-74), a process that depends on PI-3 kinase activation (Sparks, J. D. et al. (1996) Biochem J 313(Pt 2), 567-74; and Phung, T. L. et al. (1997) J Biol Chem 272(49), 30693-702). Therefore, to determine if similar signaling is also involved in the effects of Ω-3 fatty acids, rat primary hepatocytes were treated for 5 hours, in the absence or presence of the PI-3 kinase inhibitor wortmannin (1 mM), with BSA alone or BSA complexed with DHA or OA. Then, the total apoB mass that had accumulated in the conditioned medium of each well was determined by radioimmunoassay (Methods). As shown in FIG. 12, apoB recovery was significantly decreased (P<0.01) in the DHA-treated group compared to control. Notably, the recovery of apoB was restored to the control level by wortmannnin treatment. This could not be attributed to a non-specific effect of wortmannnin on apoB recovery, as evidenced by the lack of statistical effects of wortmannin treatment in the BSA and OA groups. Wortmannin also inhibited DHA-induced degradation of apoB newly synthesized in rat primary hepatocytes, assessed in a pulse-chase analysis using [³⁵S]methionine for metabolic labeling. Overall, these results imply that Ω-3 fatty acids act via an inducible process involving PI-3 kinases.

Ω-3 Fatty Acid-Stinulated Degradation of apoB in Hepatic Cells Involves Lipid Peroxidation

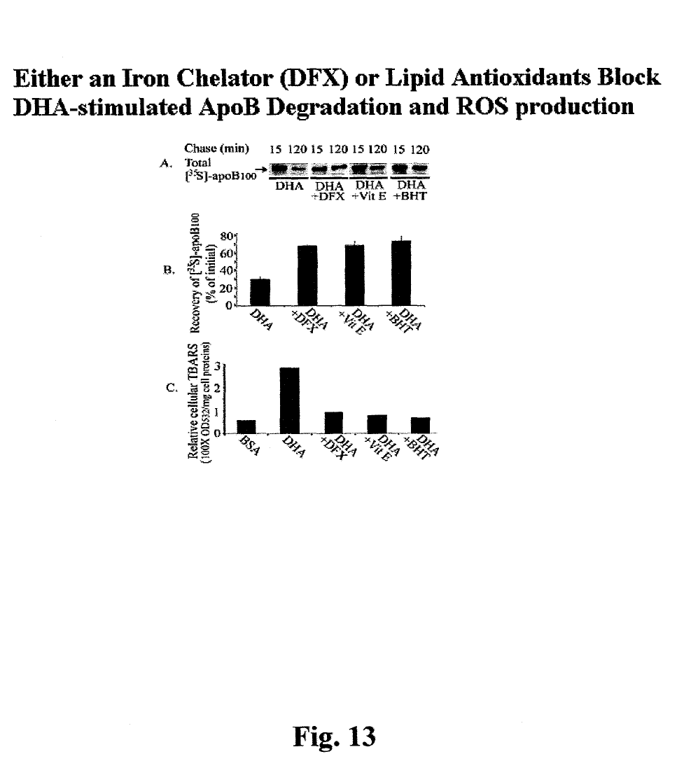

To further explore the underlying mechanisms, rat hepatoma cells or primary hepatocytes were incubated with the Ω-3 fatty acid DHA complexed to BSA, while blocking candidate cellular mediators. Inhibitors of the major classes of intracellular proteases had no significant effects on DHA-stimulated apoB₁₀₀ degradation. Ω-3 fatty acids are precursors to eicosanoids, but aspirin (which blocks their formation) had no effect. Ω-3 fatty acids can stimulate LDL receptor activity, but it is founded that primary hepatocytes from LDL receptor knock-out mice (which express no functional LDL receptors) still responded to the addition of Ω-3 fatty acids by reducing apoB secretion. Interestingly, treatment of cells with the Ca⁺⁺ chelator EGTA or BAPTA blocked DHA-stimulated apoB₁₀₀ degradation. There was, however, strong evidence against a role for calcium. Chelation of intracellular Ca⁺⁺ with BAPTA-AM, pharmacologic blockage of Ca⁺⁺ channels, depletion of the ER-calcium pool, and increasing Ca++ influx with an ionophore each had no effect. Because EGTA also chelates Fe⁺⁺⁺, and DHA is subject to Fe⁺⁺⁺-dependent peroxidation, the role of this lipid modification was examined. As shown in FIG. 13A and 13B, Desferroxamine (DFX), a chelator of Fe⁺⁺⁺, the antioxidant vitamin E (Vit E), and the synthetic antioxidant BHT, each inhibited DHA-stimulated apoB₁₀₀ degradation by ˜60%. In a TBARS assay (a standard index of lipid peroxidation, Stocks et al., 1974, Clin Sci Mol Med 47,215-222), as shown in FIG. 13C, DHA incubation, compared to BSA, increases reactive oxygen species (ROS), which were reduced by co-treatment with DFX, Vit E, or BHT.

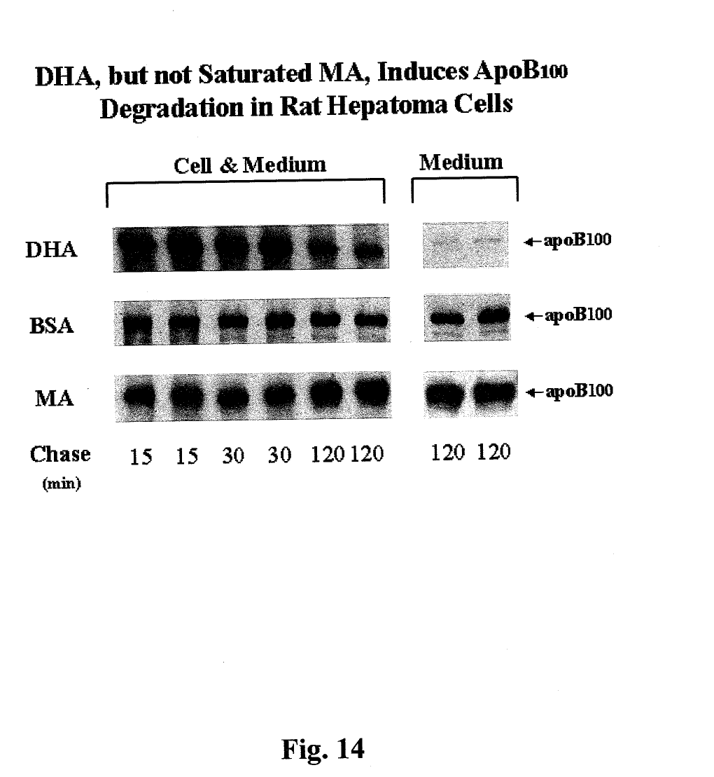

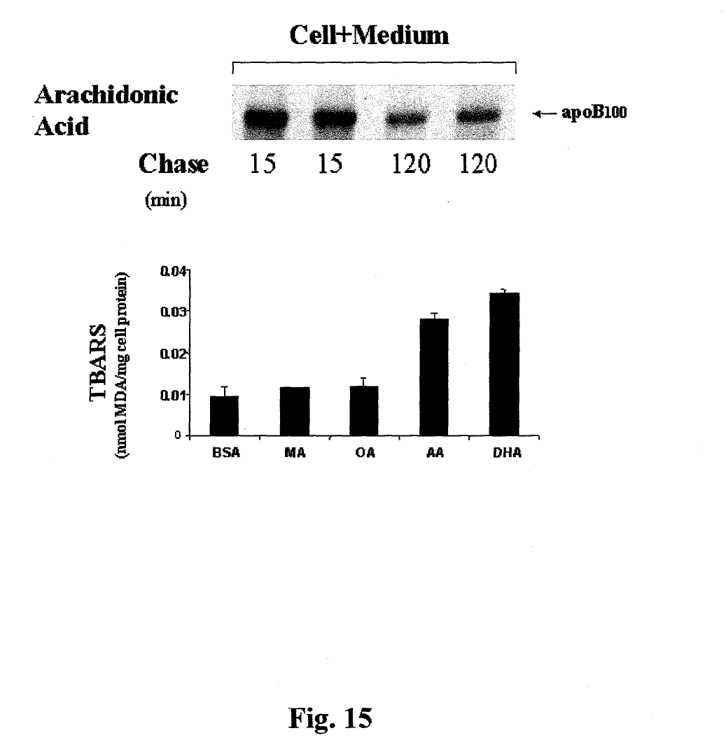

Moreover, a saturated fatty acid, myristic acid (MA), and another unsaturated fatty acid, arachidonic acid (AA), were incubated with rat hepatoma cells. As shown in FIG. 14, MA, a saturated fatty acid does stimulate apoB degradation, which is particularly apparent in the medium data. It is also shown by FIG. 15B, that MA does not increase ROS levels, similar to BSA (control) and OA (a mono-unsaturated fatty acid has been shown not to induce apoB₁₀₀ degradation). FIGS. 15A and 15B, shows that AA, an unsaturated fatty acid, stimulates the degradation of apoB, as well as increases the ROS level.

Therefore, DHA-stimulated degradation of apoB₁₀₀ is a post-ER process involving PI3 kinase and also lipid peroxidation, leading to modifications of apoB₁₀₀ and its destruction by a novel proteolytic mechanism.

ROS Regulates Basal ApoB Degradation in Primary Hepatocytes

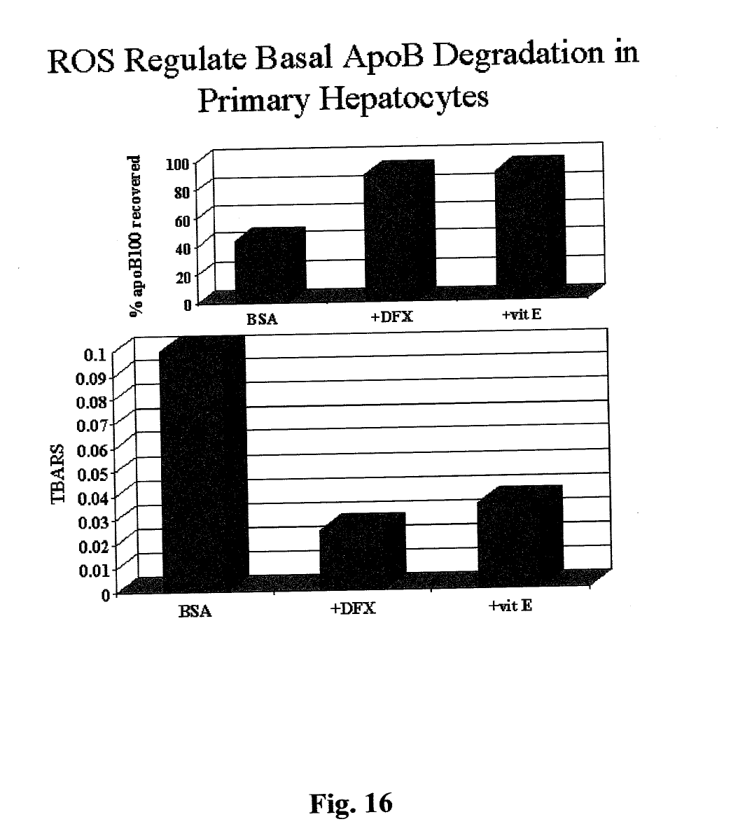

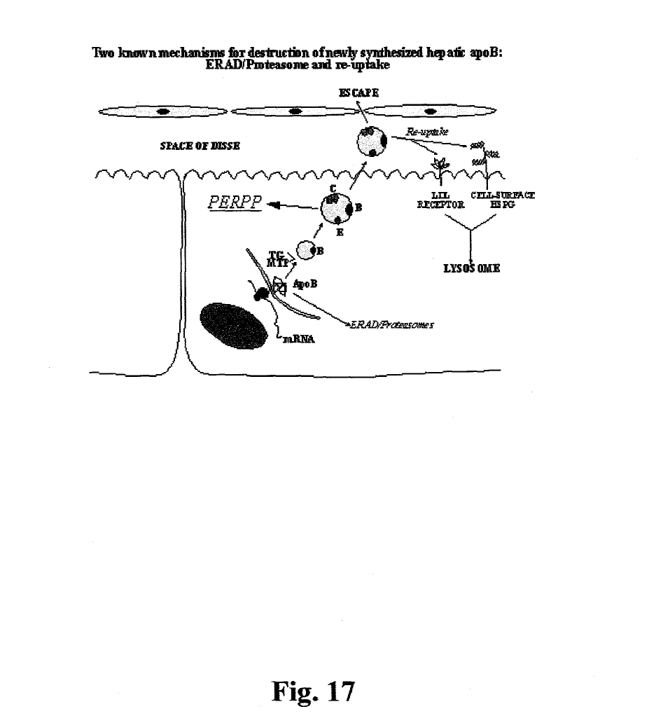

Primary rat hepatic cells were also incubated with DFX or Vit E complexed with BSA, and the percentage of apoB₁₀₀ recovery and ROS were measured. As shown in FIG. 16A, at the basal state (BSA), there is degradation of about half of the apoB pool, and this basal level of degradation is essentially reversed by treatment with DFX and Vit E. In addition, incubation with DFX or Vit E results in reduced ROS level. (FIG. 16B) Thus, the contribution of ERAD to degradation of newly synthesized apoB in hepatocytes must be quite minor under physiologically relevant conditions, i.e., basal degradation under these conditions is mediated by a pathway regulated by ROS, consistent with PERPP having the major role in regulating apoB secretion.

Discussion

The results disclosed herein establish that the treatment of hepatic cells with Ω-3 fatty acids induces the degradation of newly synthesized apoB through a process that is distinct from the two previously described degradative pathways, ERAD and re-uptake. This third “threat” to nascent apoB falls somewhere between the other processes, which are at the two temporal extremes of the secretory pathway. The discussion to follow will focus on the intrinsic characteristics of the Ω-3 fatty acid-induced process and its potential to regulate apoB-lipoprotein production in different physiologic and pathophysiologic states.

The Substrates of Ω-3 Fatty Acid-induced Degradation

Recent data (summarized in Davis, R. A. (1999) Biochim Biophys Acta 1440(1), 1-31 and Rustaeus, S. et al. (1999) J Nutr 129(2S Suppl), 463S-466S) support the model that a small apoB-containing lipoprotein of HDL density is the “primordial” particle that results from the MTP-dependent phase of initial lipidation in the ER. These primordial particles can be secreted directly as HDL-density lipoproteins or they can be further lipidated within the cell to VLDL density, and then secreted. Because apoE associates with nascent VLDL particles after apoB does (Fazio, S. et al. (1992) Journal of Biological Chemistry 267, 6941-6945; Fazio, S., and Yao, Z. (1995) Arteriosclerosis and Thrombosis 15, 593-600; and Janero, D. R., and Lane, M. D. (1983) Journal of Biological Chemistry 258, 14496-14504) and may promote VLDL triglyceride secretion (Mensenkamp, A. R. et al. (1999) J Biol Chem 274(50), 35711-8 and Tsukamoto, K. et al. (2000) J Lipid Res 41(2), 253-9), the association of nascent VLDL with apoE probably occurs during the conversion of primordial apoB-lipoproteins to more lipidated particles. Thus, the relative refractoriness of apoB associated with lipoproteins more dense than VLDL to degradation stimulated by DHA or EPA (FIGS. 1 and 2) and the global effects of DHA and EPA on apoE and the other VLDL apoproteins (FIGS. 3 and 4; Table 1) indicate that Ω-3 fatty acids most likely stimulate the degradation of apoB that has already been assembled into primordial particles, and presumably after at least some maturation of these primordial particles by association with apoE. This implies that the substrate, like that for re-uptake, is lipoprotein-associated apoB, in contrast to ERAD, which targets apoB to degradation prior to significant particle assembly.

The D-3 Fatty Acid-induced Degradation of Newly Synthesized apoB Occurs in a Post-ER Compartment or Compartments

In contrast to ERAD, Ω-3 fatty acid-induced proteolysis appears to be a post-ER event, given the effect of DHA on the recovery of apoB₁₀₀ from the Golgi (FIG. 10) and the protection afforded by brefeldin A (FIG. 11). In contrast to cell-surface re-uptake, Ω-3 fatty acids do not exert their effects through an interaction of newly exported lipoproteins with receptors on the plasma membrane (FIG. 9). Thus, our data indicate that the Ω-3 effect occurs after the ER but before the particles exit the cell. For convenience, the pathway of hepatic apoB degradation induced by Ω-3 fatty acids is referred to as “post-ER pre-secretory proteolysis” or PERPP (Williams, K. J. et al. (2001) Current Opinion in Lipidology 12(2)).

The Mechanisms Involved in Post-ER Proteolysis of apoB

Certainly, proteases are present in post-ER compartments (Neurath, H. (1989) in Proteolytic Enzymes (Benyon, R. J., and Bond, J. S., eds), pp. 1-13, IRL Press, Oxford), including the Golgi apparatus (Molloy, S. S. et al. (1999) Trends Cell Biol 9(1), 28-35). Moreover, Cartright et al. have demonstrated degradation of apoB in Golgi fractions isolated from rabbit hepatocytes (Cartwright, I. J., and Higgins, J. A. (1996) Biochemical Journal 314, 977-984), consistent with degradation by proteases in that organelle. The actual signal that identifies apoB₁₀₀ or apoB₄₈ for post-ER proteolysis in the presence of Ω-3 fatty acids is unknown, but several features can be inferred from the data disclosed herein. It is possible that the signal responsible for targeting apoB-containing lipoproteins in cells incubated with Ω-3 fatty acids is established before the particles exit from the ER and serves to trigger the appropriate trafficking of the doomed substrate to post-ER degradation. Along these lines, it has been suggested that insulin-stimulated apoB degradation in rat primary hepatocytes involves PI-3 kinase translocation to the ER membrane and the sorting of apoB to a post-ER degradative pathway (Phung, T. L. et al. (1997) J Biol Chem 272(49), 30693-702). ApoB degradation may be induced by Ω-3 fatty acids through a similar process, based on the striking features both metabolic perturbations have in common; i.e., both stimulate degradation that 1) preferentially decreases the secretion of apoB associated with large, buoyant lipoproteins (FIG. 1 and references (Sparks, J. D., and Sparks, C. E. (1994) Biochimica et Biophysica Acta 1215, 9-32 and Sparks, J. D., and Sparks, C. E. (1990) J Biol Chem 265(15), 8854-62)), 2) resists proteasome inhibitors (FIG. 8 and J. Sparks, unpublished studies), 3) occurs post-ER (FIGS. 10 and 12 and reference (Sparks, J. D. et al. (1996) Biochem J 313(Pt 2), 567-74)), and 4) is inhibited by wortmannin (FIG. 12 and references (Sparks, J. D. et al. (1996) Biochem J 31 3(Pt 2), 567-74 and Phung, T. L. et al. (1997) J Biol Chem 272(49), 30693-702)). Other possible effects of fish oils that could occur within the ER include competition with palmitate or myristate for fatty acylation of proteins, such as apoB (Hoeg, J. M. et al. (1988) J Lipid Res 29(9), 1215-20 and Kamanna, V. S., and Lee, D. M. (1989) Biochem Biophys Res Commun 162(3), 1508-14), or incorporation into eicosanoids that have specific metabolic effects on protein targeting or degradation (James, M. J. et al. (2000) Am J Clin Nutr 71(1 Suppl), 343S-8S). Of interest, palmitoylation of apoB in the ER of McA hepatoma cells is required for the proper intracellular sorting of lipoproteins to the Golgi (Zhao, Y. et al. (2000) Mol Biol Cell 11(2), 721-34).

The other possibility is that the signal that identifies apoB for PERPP occurs after the protein exits from the ER. For example, Ω-3 fatty acids incorporated into VLDL-phospholipids as part of post-ER remodeling (Vance, J. E., and Vance, D. E. (1990) Annual Review of Nutrition 10, 337-356 and Bamberger, M. J., and Lane, M. D. (1990) Proceedings of the National Academy of Sciences 87, 2390-2394) could affect apoB conformation (e.g., Kleinman, Y. et al. (1988) J Lipid Res 29(6), 729-43; Banuelos, S. et al. (1995) J Biol Chem 270(16), 9192-6; Ziegler, O. et al. (1996) Diabetes Metab 22(3), 179-84; and Chauhan, V. et al. (1998) Biochemistry 37(11), 3735-42) and might alter interactions of the nascent particles with other components of the secretory pathway to provoke degradation. Along these lines, deficient phospholipid biosynthesis produced by choline deprivation stimulates apoB degradation by a process that appears remarkably similar to Ω-3-induced PERPP in that it affects mainly large VLDL-like particles and occurs post-ER (Yao, Z., and Vance, D. E. (1988) Journal of Biological Chemistry 263, 2998-3004; Verkade, H. J. et al. (1993) J Biol Chem 268(33), 24990-6; and Vermeulen, P. S. et al. (1997) J Lipid Res 38(3), 447-58).

Nevertheless, as disclosed herein, the TBARS assay demonstrates that DHA-stimulation of apob₁₀₀ degradation is associated with an elevated level of reactive oxygen species (ROS). While, DFX (which blocks iron-mediated lipid peroxidation) and two other antioxidants, Vit E and BTH, each inhibit DHA-stimulated apoB₁₀₀ degradation, and reduce the elevated ROS level that was stimulated by DHA. (FIG. 13) Therefore, DHA-stimulated degradation of apoB₁₀₀ is a post-ER process involving PI-3 kinase and also lipid peroxidation, leading to modifications of apoB100 and its destruction by a novel proteolytic mechanism.

The General Significance of PERPP In Vivo

Based on many experimental studies, the major determinant of hepatic apoB secretion in vivo is considered to be pre-secretory degradation (for recent reviews, see Yao, Z. et al. (1997) J Lipid Res 38(10), 1937-53; Davis, R. A. (1999) Biochim Biophys Acta 1440(1), 1-31; Williams, K. J., and Fisher, E. A. (2001) Current Opinion in Lipidology 12(2); and Sparks, J. D., and Sparks, C. E. (1994) Biochimica et Biophysica Acta 1215, 9-32). ERAD can be provoked by lipid deficiency, but this seems an unlikely candidate to make significant contributions to apoB degradation under physiologic conditions in which neither hepatic MTP activity nor plasma fatty acid levels are below those found to induce ERAD in cell culture models. (e.g., Dixon, J. L. et al. (1991) Journal of Biological Chemistry 266, 5080-5086 and Liang, J. et al. (1998) J Biol Chem 273(52), 35216-21.) This is consistent with the data, as disclosed herein, that about half of the apoB pool is degraded in the basal state, and that this basal level of degradation is essentially reversed by DFX and Vit E. (FIG. 16A) Moreover, the apoC-III transgenic mouse provides a specific example of increased fatty acid availability in vivo, but without any increase in hepatic apoB secretion (Aalto-Setala, K. et al. (1992) J Clin Invest 90(5), 1889-900).

Regarding the two other “threats”, re-uptake and PERPP, there are recent data to support their roles as regulators of net hepatic production of apoB-lipoproteins in vivo. In support of re-uptake, animals lacking LDL receptors exhibit increased hepatic production of VLDL (Horton, J. D. et al. (1999) J Clin Invest 103(7), 1067-76 and Twisk, J. et al. (2000) J Clin Invest 105(4), 521-32), consistent with the inventors' findings in cell culture (Williams, K. J. et al. (1990) Journal of Biological Chemistry 265, 16741-16744). Furthermore, increased expression of bridging molecules, leading to enhanced apoB re-uptake via HSPGs, may account for some of the hypolipidemic effects of fibric acid derivatives in vivo (Williams, K. J. et al. (1992) J Biol Chem 267(19), 13284-92 and Williams, K. J. et al. (1991) Journal of Clinical Investigation 88,1300-1306).

In support of PERPP, Ω-3 fatty acid-enriched diets consistently lower VLDL levels in human subjects (Harris, W. S. (1989) J Lipid Res 30(6), 785-807), and the mechanism appears to be decreased hepatic VLDL production (Huff, M. W., Telford, D. E., and Barrett, P. H. (1992) Arterioscler Thromb 12(8), 902-10). Given the similarities outlined above between PERPP stimulated by Ω-3 fatty acids and apoB degradation stimulated by insulin, it is speculated that syndromes of insulin resistance should reduce PERPP in vivo, thereby resulting in the overproduction of large buoyant apoB-lipoproteins. Thus, PERPP may contribute to the pathogenesis of familial combined hyperlipidemia (FCHL), syndrome X, and the metabolic syndrome (for recent reviews, see Brunzell, J. D., and Hokanson, J. E. (1999) Diabetes Care 22 Suppl 3, C10-3 and Ginsberg, H. N. (2000) J Clin Invest 106(4), 453-8), as well as the syndrome of hyperlipidemia and insulin resistance seen after administration of HIV protease inhibitors (Carr, A. et al. (1998) Aids 12(7), F51-8; Vigouroux, C. et al. (1999) Diabetes Metab 25(3), 225-32; and Mulligan, K. et al. (2000) J Acquir Immune Defic Syndr 23(1), 35-43). Consistent with this model, the acute post-prandial rise in insulin levels is associated with a specific decrease in hepatic VLDL production in vivo (Chirieac, D. V. et al. (2000) Am J Physiol Endocrinol Metab 279(5), E1003-11), and insulin-resistant animals were recently reported to exhibit decreased degradation of newly synthesized apoB (Taghibiglou, C. et al. (2000) J Biol Chem 275(12), 8416-25).

In conclusion, PERPP is a distinct step in the regulation of apoB secretion from hepatocytes in vitro, with a plausible role in vivo as well. Identification of physiologic stimuli for PERPP, the cellular mechanisms for targeting of apoB to this degradative pathway, and the protease(s) involved should advance our understanding of the regulation of hepatic lipoprotein production in both normal and pathophysiological states and may provide new targets for pharmacologic intervention.

While this invention has been described with a reference to specific embodiments, it will be obvious to those of ordinary skill in the art that variations in these methods and compositions may be used and that it is intended that the invention may be practiced otherwise than as specifically described herein. Accordingly, this invention includes all modifications encompassed within the spirit and scope of the invention as defined by the claims.

What is claimed is:

1. A method of treating a patient, comprising administering a therapeutically effective amount of a Therapeutic to said patient, wherein said Therapeutic acts for a period of time to lower plasma concentrations of apoB and/or an apoB-containing lipoprotein and/or a component of an atherogenic lipoprotein by stimulating post-ER pre-secretory proteolysis (PERPP).

2. The method of claim 1, wherein said patient has a cardiovascular disorder and/or a hyperlipidemia.

3. The method of claim 2, wherein said cardiovascular disorder and/or hyperlipidemia is selected from the group consisting of angina, atherosclerosis, restenosis, claudication, unstable angina, stroke, transient ischemic attacks, coronary artery disease, peripheral vascular disease, cerebral vascular disease, endothelial dysfunction, elevated plasma concentration of LDL, elevated plasma concentration of a VLDL, elevated plasma concentration of a lipoprotein(s), elevated plasma concentration of an apoB-containing lipoprotein, elevated plasma concentration of a beta-VLDL, elevated plasma concentration of an atherogenic lipoprotein, and a syndrome recommended for treatment by the Adult Treatment Panel of the National Cholesterol Education Program.

4. The method of claim 1, wherein said patient has a metabolic disorder or syndrome.

5. The method of claim 4, wherein said metabolic disorder or syndrome is selected from the group consisting of hypercholesterolemia, hyperlipidemia, hyper lipoproteinemia, familial hyperlipidemia, familial combined hyperlipidemia, syndrome X, insulin resistance syndromes, diabetes, secondary hyperlipidemia, secondary insulin resistance, hyperlipidemia and/or insulin resistance that is secondary to a medication, hyperlipidemia and/or insulin resistance that is secondary to an anti-HIV medication, and metabolic disorders associated with vascular disease.

6. The method of claim 1, wherein said Therapeutic is employed in combination with another type of cholesterol lowering drug.

7. The method of claim 6, wherein the other cholesterol lowering drug is selected from the group consisting of an HMG CoA reductase inhibitor, a squalene synthetase inhibitor, a fibric acid derivative, probucol, a bile acid sequestrant, nicotinic acid and neomycin.

8. The method of claim 7, wherein said HMG CoA reductase inhibitor is selected from the group consisting of pravastatin, lovastatin, simvastatin, atorvastatin, fluvastatin and cerivastatin.

9. The method of claim 7, wherein said fibric acid derivative is selected from the group consisting of gemfibrolzil, fenofibrate, clofibrate, bezafibrate, ciprofibrate, and clinofibrate.

10. The method of claim 1, wherein said Therapeutic is a PI-3 kinase inducer.

11. The method of claim 1, wherein said Therapeutic lowers hepatic content of an anti-oxidant.

12. The method of claim 1, wherein said Therapeutic lowers hepatic content of thioredoxin.

13. The method of claim 1, wherein said Therapeutic lowers hepatic content of vitamin E.

14. The method of claim 1, wherein said Therapeutic inhibits transfer of vitamin E.

15. The method of claim 1, wherein said Therapeutic inhibits transfer of vitamin E by a phospholipid transfer protein.

16. The method of claim 1, wherein said Therapeutic induces reactive oxygen species (ROS).

17. A method of screening a pharmaceutical compound, wherein said compound stimulates a PERPP that occurs in a post-ER compartment or compartments, comprising the steps of
a) contacting hepatic cells with said compound; and
b) measuring cellular content and/or secretion levels of an apoB, an apoB-containing lipoprotein and/or a component of an atherogenic lipoprotein to determine efficacy of said compound,
wherein said method further comprises at least one of the steps of
determining that a proteosome inhibitor does not substantially impair the efficacy of said compound;
determining that heparin does not substantially impair the efficacy of said compound;
determining that the efficacy of said compound is substantially reversible by an antioxidant;
determining that the efficacy of said compound is substantially reversible by a PI-3 kinase inhibitor;
determining that said compound does not substantially affect apoB level in the ER; and
determining that said compound substantially increases cellular content of reactive oxygen species (ROS).

18. The method of claim 17, wherein said proteosome inhibitor is selected from the group consisting of lactacystin, MG132, ALLN, vinyl sulfones, and analogs or derivatives thereof.

19. The method of claim 17, wherein said antioxidant is selected from the group consisting of Vitamin E, desferroxamine (DFX), butylhydroxytoluene (BHT), EGTA, a lipid antioxidant, and an iron chelator.

20. The method of claim 17, wherein said PI-3 kinase inhibitor is wortmannin or LY compound (290004).

21. A method of screening for at least one gene, wherein said least one gene or RNA(s) or protein(s) encoded thereof regulate intracellular apoB degradation through PERPP, comprising the steps of
a) preparing genetically engineered hepatic cells containing a mutation that affects said at least one gene; and
b) measuring cellular content and/or secretion levels of an apoB, an apoB-containing lipoprotein, and/or a component of an atherogenic lipoprotein from said genetically engineered hepatic cells to determine that said at least one gene or said RNA(s) or protein(s) encoded thereof regulate said intracellular apoB degradation,
wherein said method further comprises at least one of the steps of
determining that a proteosome inhibitor does not substantially impair the regulation of intracellular apoB degradation by said at least one gene or said RNA(s) or protein(s) encoded thereof;
determining that heparin does not substantially impair the regulation of intracellular apoB degradation by said at least one gene or said RNA(s) or protein(s) encoded thereof;
determining that said genetically engineered hepatic cells exhibit substantially elevated or reduced apoB level in Golgi, but not ER; and
determining that said genetically engineered hepatic cells exhibit substantially elevated or reduced cellular content of ROS.

22. A method of screening an HIV protease inhibitor, which exhibits reduced or absent inhibition of intracellular apoB degradation through PERPP, comprising the steps of
a) contacting hepatic cells which exhibit PERPP with said HIV protease inhibitor;
b) measuring cellular content and/or secretion levels of apoB, an apoB-containing lipoprotein and/or a component of an atherogenic lipoprotein to determine said reduced or absent inhibition of intracellular apoB degradation by said HIV protease inhibitor.

23. A method of treating an HIV-infected individual by administering a therapeutically effective amount of an HIV protease inhibitor, which inhibitor exhibits reduced or absent inhibition of intracellular apoB degradation.

24. A method of treating an HIV-infected individual by administering a first therapeutically effective amount of an HIV protease inhibitor and a second therapeutically effective amount of a compound, wherein said compound acts for a period of time to lower plasma concentrations of apoB, an apoB-containing lipoproteins, and/or a component of an atherogenic lipoprotein by stimulating PERPP.
